(19) United States
(12) Patent Application Publication
(10) Pub. No.: US 2004/0137588 A1
(43) Pub. Date: Jul. 15, 2004

(54) Purification of vascular endothelial growth factor-b

(76) Inventors: Sergio D.B. Scrofani, Victoria (AU); Andrew D. Nash, Victoria (AU); Louis J. Fabri, Richmond (AU); Andrew W. Mackenzie, Victoria (AU); Pierre D. Scotney, Victoria (AU)

Correspondence Address: Edward W Grolz, Scully Scott Murphy & Presser, 400 Garden City Plaza, Garden City, NY, 11530, US

(21) Appl. No.: 10/204,070

(22) PCT Filed: Feb. 16, 2001
(86) PCT No.: PCT/AU01/00160

(30) Foreign Application Priority Data
Feb. 16, 2000 (AU) PQ 5681

(51) Int. Cl. C12N 9/00

(52) U.S. Cl. 435/183; 530/413

(57) ABSTRACT

The present invention provides a method for purifying recombinant peptides, polypeptides or proteins away from truncated or other full-length forms of these molecules. In particular the invention contemplates a method of purifying a vascular endothelial growth factor (VEGF) molecule by subjecting a biological sample containing the molecule to be purified to affinity chromatography under conditions sufficient for the full length molecules to bind and not the truncated or clipped forms. In the preferred embodiment there are two columns, the first is based on affinity for a poly his tag, the second column based on heparin binding affinity. Particularly preferred VEGF molecules are untagged VEGF-B₁₆₇, hexa-His-tagged VEGF-B₁₆₇, hexa-His-tagged VEGF-B₁₈₆ and hexa-His-tagged VEGF-B₁₀₋₁₀₈.

### FIELD OF THE INVENTION

The present invention relates generally to a method of producing recombinant peptides, polypeptides and proteins. More particularly, the present invention provides a method of purifying recombinant peptides, polypeptides or proteins away from truncated or other non-full length forms of these molecules. Even more particularly, the present invention contemplates a method of purifying a vascular endothelial growth factor (VEGF) molecule or a derivative or homologue thereof including amino acid tagged forms or other peptide, polypeptide or protein by subjecting a preparation containing the molecule to be purified to affinity chromatography under chromatographic conditions sufficient for full length molecules but not truncated or non-full length molecules corresponding to said full length molecules to bind or otherwise associate by the affinity process. In a preferred embodiment, the purification involves optionally subjecting a preparation containing the molecule to be purified to an affinity column based on the properties of an exogenous amino acid sequence followed by a second affinity column based on properties inherent with the peptide, polypeptide or protein. The present invention is further directed to a peptide, polypeptide or protein such as a VEGF molecule or a derivative or homologue thereof purified by the methods of the present invention. Particularly preferred VEGF molecules are VEGF-B molecules including untagged VEGF-B₁₆₇, hexa-His-tagged VEGF-B₁₆₇, hexa-His-tagged VEGF-B₁₈₆ and hexa-His-tagged VEGF-B₁₀₋₁₀₈.

### BACKGROUND OF THE INVENTION

Reference to any prior art in this specification is not, and should not be taken as, an acknowledgment or any form of suggestion that this prior art forms part of the common general knowledge in Australia or any other country.

Bibliographic details of the publications referred to by anthor in this specification are collected at the end of the description.

Recombinant DNA technology provides the means for the production of peptides, polypeptides and proteins in large quantity. This is especially required for molecules required for therapeutic interventionist purposes where vast quantities are required. However, the molecules also need to be highly purified.

Cytokines and growth factors are important molecules for which many are available in recombinant form. However, despite the available knowledge as to their structure and function, the therapeutic use of such molecules will depend upon the level of purity which can be obtained.

One particularly important growth factor is vascular endothelial growth factor (hereinafter referred to as “VEGF”). This molecule is also known as vasoactive permeability factor. VEGF is a secreted, covalently linked homodimeric glycoprotein that specifically activates endothelial tissues (Senger et al., 1993). A range of functions have been attributed to VEGF such as its involvement in normal angiogenesis including formation of the corpus luteum (Yan et al., 1993) and placental development (Sharkey et al., 1993), regulation of vascular permeability (Senger et al., 1993), inflammatory angiogenesis (Sunderkotter et al., 1994) and autotransplantation (Dissen et al., 1994) and human diseases such as tumour promoting angiogenesis (Folkman & Shing, 1992), rheumatoid arthritis (Koch et al., 1994) and diabetes related retinopathy Wolkman & Shing, 1992).

Based on a high level of sequence homology within a region incorporating 8 equally spaced cysteine residues (cystine knot motif/VEGF homology domain), four further proteins can be included within the VEGF family: placenta growth factor (PLGF), VEGF-B, VEGF-C and VEGF-D. Compared to VEGF-A relatively little is known about methods of production for these other members of the VEGF family. The five members of the family are now known to interact differentially with 3 distinct receptor tyrosine kinases. While VEGF-A binds VEGFR1 and R2, PLGF and VEGF-B bind only to VEGFR1. In contrast VEGF-C and D bind VEGFR2 and, in addition, a third receptor (VEGFR3 or Flt4) restricted to lymphatic endothelium. The functional significance of the distinct receptor binding characteristics of the additional family members remains unclear. The issue of functional activity of distinct family members is further complicated by their ability to form heterodimers when co-expressed in mammalian cells.

Like VEGF-A, VEGF-B is, therefore, an important molecule and may have utility as a therapeutic agent if it can be produced and purified to a sufficiently high level. VEGF-B comprises a series of isoforms and truncated isoforms, some of which retain the receptor binding domain. Examples of VEGF-B isoforms include VEGF-B₁₆₇, VEGF-B₁₈₆ and VEGF-B₁₀₋₁₀₈. Due to a number of technical obstacles, VEGF-B isoforms have not previously been purified to near homogeneity as a homodimer and shown to be active.

VEGF-B is a member of the cystine knot family of cytokines that exhibit complex secondary structure elements, which include inter- and intra-molecular disulfide bonds. An ideal method of producing such complex eukaryotic proteins involves expression in a mammalian system, where it is likely that the protein will adopt its native conformation. However, mammalian systems produce endogenous VEGF family members, in particular VEGF-A, which form heterodimers with the expressed VEGF-B. Such heterodimers are difficult to separate from the desired homodimers and any such step would add substantially to the cost of production. An alternative method of producing pure homodimeric VEGF-B involves expression in non-mammalian systems such as Escherichia coli, where the protein is expressed most commonly as inclusion bodies. Inclusion bodies can in general only be solubilized under harsh denaturing conditions and proteins produced in such a way must be refolded into the correct conformation. For proteins with complex secondary structure, such as VEGF-B, this can create problems during refolding such that incorrectly folded and inactive proteins can result. Consequently, specific refolding conditions are required for VEGF-B. In addition to complex secondary structure, the hydrophobic nature of VEGF-B, and VEGF-B₁₆₇ in particular, leads to aggregation during refolding and purification and this can result in complete loss of protein. This issue requires particular attention during purification. One further complication with some conventional purification techniques applied to VEGF-B is the inability to discriminate between full length VEGF-B molecules and truncated or “clipped” variants. Consequently, during refolding, hybrids can form between a full length molecule and a truncated variant leading to an inactive molecule or a molecule exhibiting undesirable properties.

The present invention describes a strategy that overcomes these technical obstacles to yield highly purified homodimeric VEGF-B isoforms that have demonstrated receptor binding characteristics. The molecules purified by the present invention are particularly useful in therapeutic protocols and in diagnostic assays.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier, i.e. <400>1, <400>2, etc. A sequence listing is provided after the claims.

One aspect of the present invention provides a method of purifying a peptide, polypeptide or protein from a biological sample said method comprising subjecting the biological sample to affinity chromatography comprising an affinity matrix under chromatographic conditions sufficient for the fill length but not a truncated or non-fill length peptide, polypeptide or protein corresponding to said full length peptide, polypeptide or protein to be bound to or otherwise associate with the affinity matrix and then eluting said bound or associated peptide, polypeptide or protein from the affinity matrix and collecting same.

Another aspect of the present invention is directed to a method of purifying a recombinant peptide, polypeptide or protein from a biological sample said method comprising subjecting said biological sample to affinity chromatography comprising an affinity matrix which has affinity for an N-terminal or C-terminal region of said peptide, polypeptide or protein but substantially not for the N-terminal or C-terminal region of a truncated. or clipped form of said peptide, polypeptide or protein, said affinity chromatography being under chromatographic conditions sufficient to permit binding or association of full length but not truncated or non-fall length. peptide, polypeptide or protein, and then eluting the bound or associate peptide, polypeptide or protein from the affinity matrix and collecting same.

Yet another aspect of the present invention provides a method of purifying a peptide, polypeptide or protein from a biological sample comprising subjecting said biological sample to an optional first affinity chromatography comprising an affinity matrix which binds or associates said peptide, polypeptide or protein based on affinity to an N-terminal or C-terminal portion of said molecule, eluting off said bound or otherwise associated peptide, polypeptide or protein and subjecting same to a second affinity chromatography based on affinity or association with the other of an N-terminal or C-terminal portion of said molecule and eluting the peptide, polypeptide or protein bound or associated following said second affinity chromatography and collecting same.

Still yet another aspect of the present invention contemplates a method of purifying a full length VEGF-B isoform or a related polypeptide from a biological sample, said method comprising subjecting said biological sample to a first optional affinity chromatography comprising an affinity matrix based on affinity binding to multiple contiguous exogenous histidine (His) residues in the N-terminal portion of said VEGF-B isoform, eluting said VEGF-B isoform bound or otherwise associated with said first affinity chromatography and subjecting said eluted VEGF-B isoform to a second affinity chromatography based on affinity of the C-terminal portion of said VEGF-B isoform to heparin or like molecule, and then eluting and collecting said VEGF-B isoform bound or otherwise associated by said second affinity chromatography based on affinity of the C-terminal portion of said VEGF-B isoform to heparin or like molecule.

Still another aspect of the present invention contemplates a method of purifying a homomultimeric polypeptide such as a homodimeric VEGF-B isoform or similar molecule from a biological sample, said method comprising subjecting said biological sample to an optional first affinity chromatography based on affinity for exogenous basic amino acids such as polyHis or hexa-His in the N-terminal portion of said polypeptide; eluting and collecting fractions containing said polypeptide, subjecting said polypeptide to a second affinity chromatography based on affinity to heparin of the C-terminal portion of said polypeptide; eluting and collecting said polypeptide; subjecting said polypeptide to refolding conditions in the presence of Guanidine HCl (GdCl) or arginine and dialyzing refolded polypeptide against acetic acid and/or other acid with similar properties; and purifying said refolded polypeptide by reversed phase chromatography.

Yet still another aspect of the present invention contemplates a method of purifying a full length VEGF-B isoform or a related polypeptide from a biological sample, said method comprising subjecting said biological sample to a first optional affinity chromatography comprising an affinity matrix based on affinity binding to multiple contiguous exogenous histidine (His) residues in the N-terminal portion of said VEGF-B isoform, eluting said VEGF-B isoform bound or otherwise associated with said first affinity chromatography and subjecting said eluted VEGF-B isoform to a cation exchange chromatography, and then eluting and collecting said VEGF-B isoform bound or otherwise associated by said cation exchange chromatography.

Another aspect of the present invention contemplates a method of purifying a homomultimeric polypeptide such as a homodimeric VEGF-B isoform or similar molecule from a biological sample, said method comprising subjecting said biological sample to an optional first affinity chromatography based on affinity for exogenous basic amino acids such as polyHis or hexa-His in the N-terminal portion of said polypeptide; eluting and collecting fractions containing said polypeptide, subjecting said polypeptide to cation exchange chromatography, eluting and collecting said polypeptide; subjecting said polypeptide to refolding conditions in the presence of Guanidine HCl (GdCl) or arginine and dialysing refolded polypeptide against acetic acid and/or other acid with similar properties; and purifying said refolded polypeptide by reversed phase chromatography.

A further aspect of the present invention provides a method for the preparation and purification of a recombinant peptide, polypeptide or protein in homomultimeric form, said method comprising culturing a microorganism or animal cell line comprising a genetic sequence encoding a monomeric form of said peptide, polypeptide or protein under conditions sufficient for expression of said genetic sequence; obtaining cell lysate, culture supernatant fluid, fermentation fluid or conditioned medium from said microorganism or animal cell line and subjecting same to a first optional affinity chromatography step based on affinity to exogenous amino acids present in the N- or C-terminal region of said peptide, polypeptide or protein, collecting fractions containing said peptide, polypeptide or protein and subjecting said fractions to a second affinity chromatography step based on affinity to an inherent property of the amino acid sequence or structure in the C-terminal portion of said polypeptide such as binding to heparin or difference in charge; said affinity chromatography being under chromatographic conditions sufficient for full length but not truncated or non-full length peptide, polypeptide or protein to be bound or otherwise associated by said affinity chromatography; eluting and collecting said full length peptide, polypeptide or protein and subjecting same to refolding conditions in the presence of GdCl and dialyzing against acetic acid or other similar acid and then purifying the refolded polypeptide by reversed phase chromatography.

Yet another aspect of the present invention is directed to the use of a recombinant peptide, polypeptide or protein purified according to the methods herein described in the manufacture of a medicament for the treatment of a disease condition or the manufacture of an agent for use in diagnosis.

### BRIEF DESCRIPTION OF THE PREFERRED EMBODIMENTS

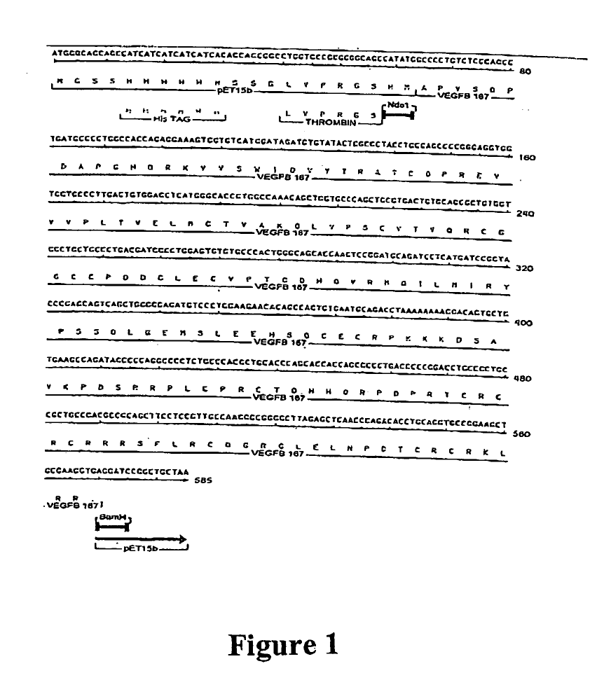

FIG. 1 is a representation of the VEGF-B₁₆₇ protein produced in E. coli and comprising a 21 amino acid leader sequence at the N-terminus and incorporating a hexa-His tag and thrombin cleavage site.

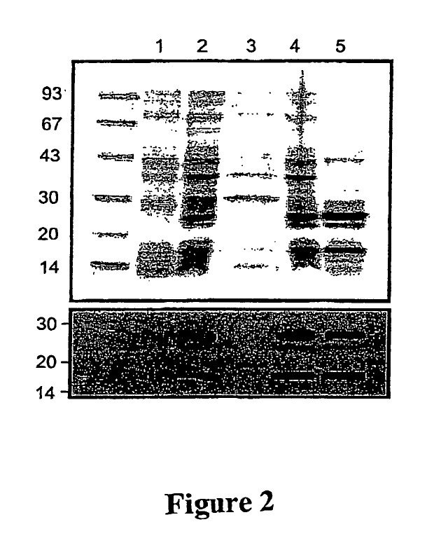

FIG. 2 is a photographic representation of an SDS-PAGE/Western Blot analysis of protein in (1) whole cells, pre-induction; (2) whole cells, post-induction; (3) soluble fraction; (4) insoluble fraction; and (5) isolated inclusion bodies of E. coli carrying the vector pET15b-VEGF-B₁₆₇.

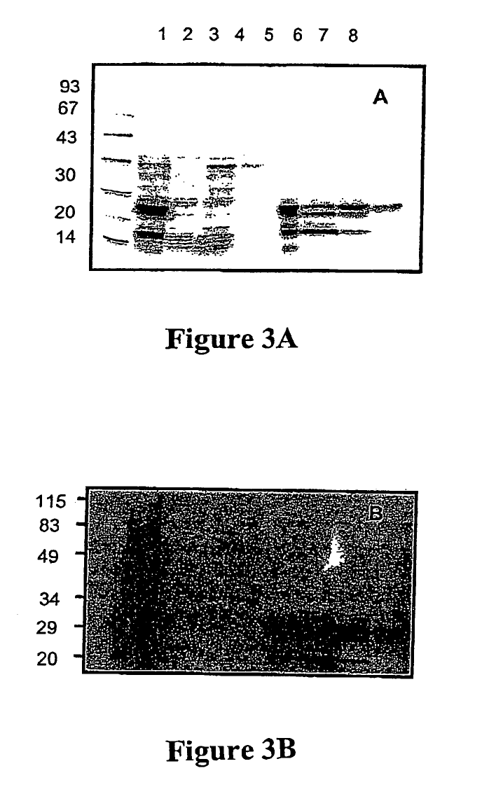

FIG. 3 is a photographic representation of an SDS-PAGE/Western Blot analysis of the eluates following (A) Reducing SDS-PAGE of nickel/heparin affinity-coomassie stain; and (B) Western blot analysis using an N-terminal VEGF-B specific antibody (1) Purified inclusion bodies (6 M GdCl, 20 mM DTT, pH 8.5) before affinity chromatography, (2) flow through (6 M GdCl, pH 8.5); (3) wash 1 (8 M urea, pH 7.5); (4) wash 2 (8 M urea, pH 6.3); (5) elution (8 M urea, 0.5 M Imidazole, pH 5.9); denaturing/reducing heparin sepharose, (6) flow through (6 M urea, 40 mM DTT, pH 8.5); (7) wash (6 M urea, 40 mM DTT, pH 8.5); (8) elution (6 M urea, 1 M NaCl, 40 m M DTT, pH 8.5).

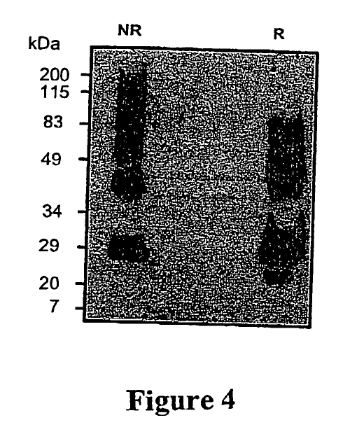

FIG. 4 is a photographic representation of nonreducing (NR) and reducing (R) forms of refolded VEGF-B₁₆₇ purified following heparin-sepharose chromatography as analyzed by SDS-PAGE and visualised by Western blot analysis.

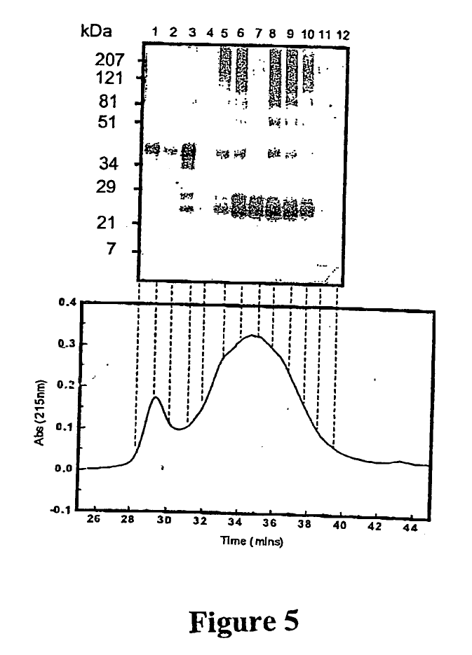

FIG. 5 is a photographic and graphical representation of fractions collected from a Brownlee C8 reversed-phase HPLC (RPHPLC) column (10×100 mm) and subjected to non-reducing SDS-PAGE.

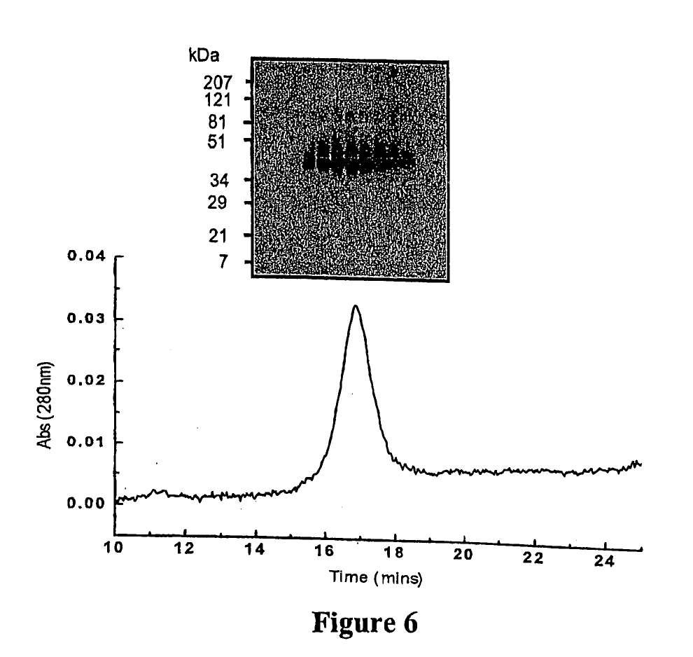

FIG. 6 is a photographic and graphical representation of pooled fractions containing predominantly dimeric VEGF-B₁₆₇ re-applied to C8 column and eluted with a linear gradient formed between 20-45% of Buffer 13 (0.12% v/v n-propanol/min).

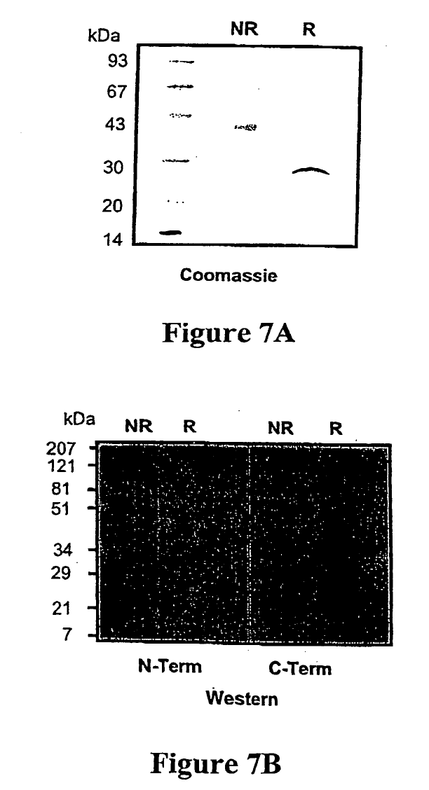

FIG. 7 is a photographic representation showing (A) Coomassie and (B) Western blot gels of VEGF-B₁₆₇ containing fractions from the C8 column of FIG. 6. [Note: N-Term refers to a polyclonal N-terminal VEGF-B peptide specific antibody and C-Term refers to a polyclonal C-terminal VEGF-B₁₆₇ peptide specific antibody].

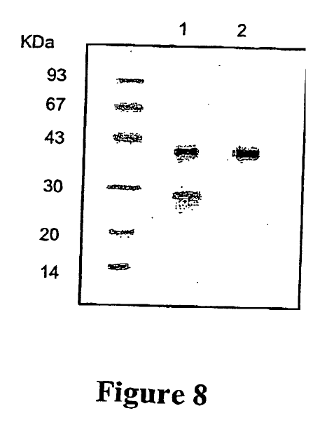

FIG. 8 is a photographic representation showing VBGF-B₁₆₇ purified by (1) C8 RPHPLC and (2) a polyhydroxyethyl, a hydrophilic column.

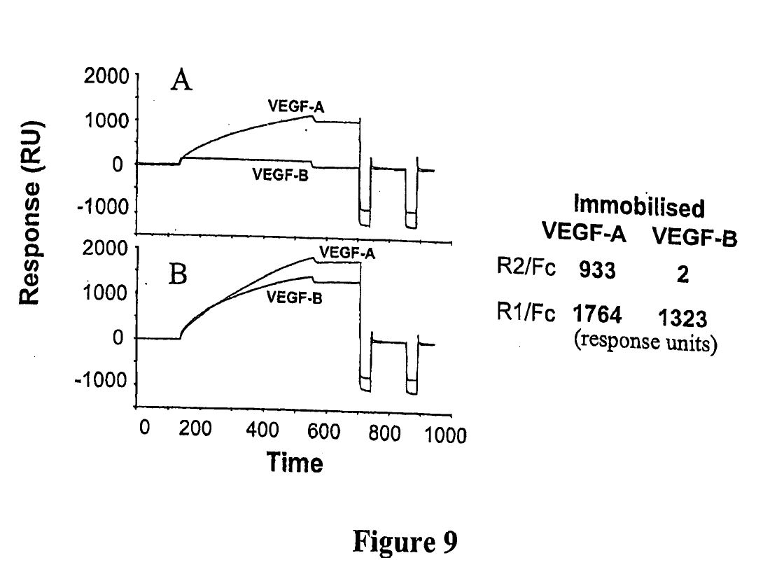

FIG. 9 is a graphical representation showing (A) biosensor analysis of binding of VEGF-A₁₆₅ or VEGF-B₁₆₇ to VEGF R2/Fc; and (B) biosensor analysis of binding of VEGF-A₁₆₅ or VEGF-B₁₆₇ to VEGF R1/Fc. Values (response units) shown represent the difference in response pre and post injection of the receptors.

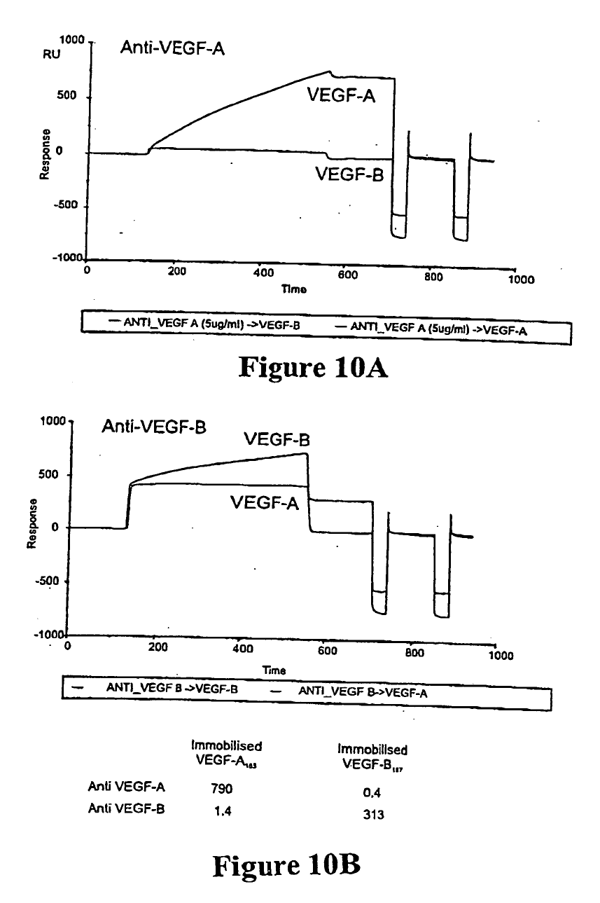

FIG. 10 is a graphical representation showing surface plasmon resonance of antibody binding to sensor chip immobilised VEGF-A₁₆₅ or VEGF-B₁₆₇.

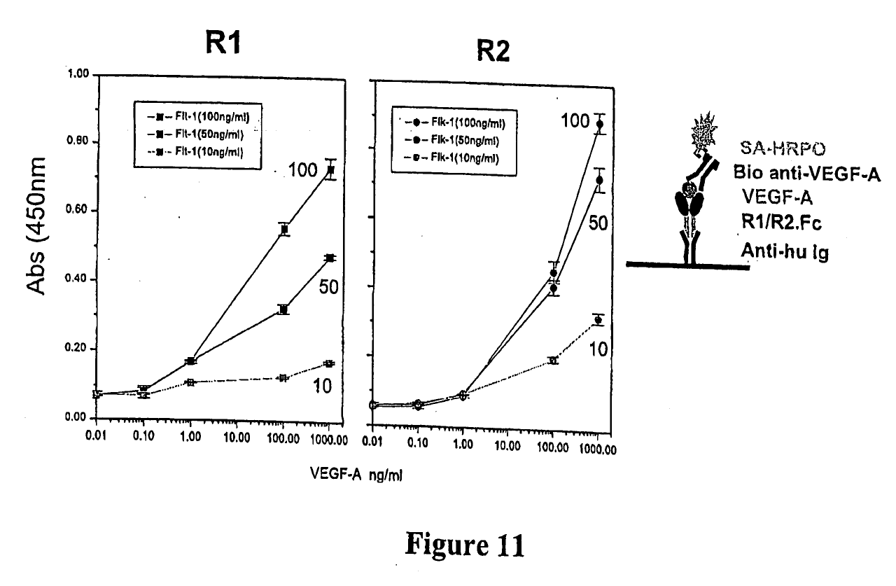

FIG. 11 is a graphical representation showing binding of VEGF-A₁₆₅ to both VEGF R1 and VEGF R2 using a range of receptor concentrations in an ELISA based system.

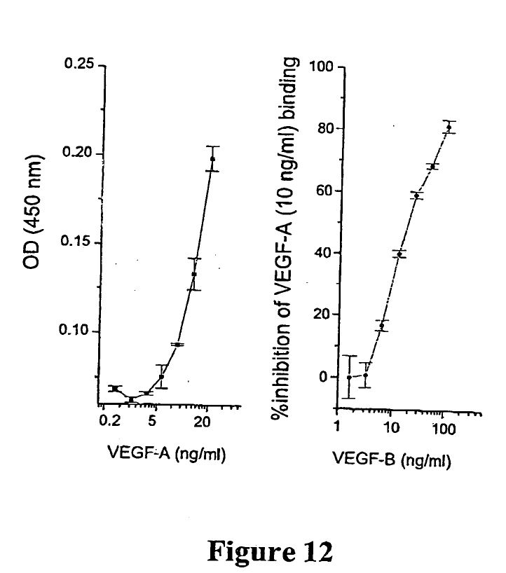

FIG. 12 is a graphical representation showing the competition of VEGF-B₁₆₇ with VEGF-A₁₆₅ for binding to VEGF R1.

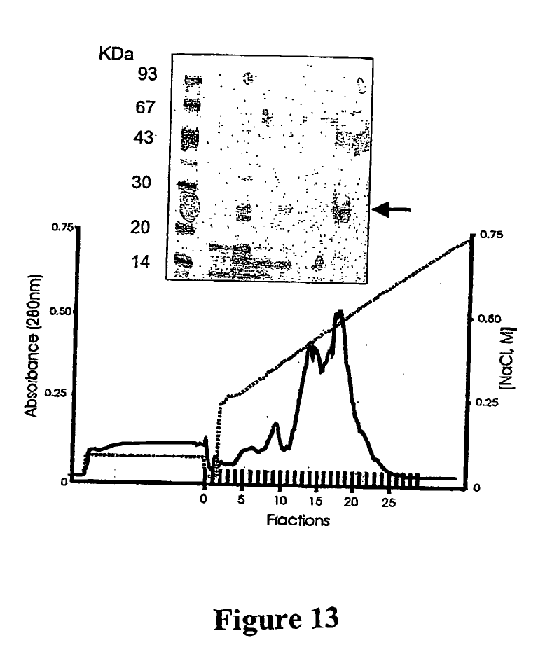

FIG. 13 is a photographic and graphical representation of the cation exchange chromatography elution profile showing the separation of full-length monomeric VEGF-B₁₆₇ (denoted by arrow) from both truncated VEGF-B₁₆₇ and contaminating proteins. The Coomassie gel above the elution profile shows the proteins contained within respective pooled fractions.

FIG. 14 is a photographic representation of non-reducing (NR) and reducing (R) forms of purified refolded His₆-VEGF-B₁₈₆ as analyzed by SDS-PAGE and visualized with Coomassie stain.

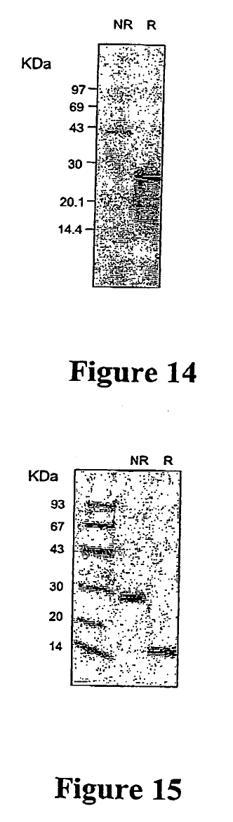

FIG. 15 is a photographic representation of non-reducing (NR) and reducing (R) forms of purified refolded His₆-VEGF-B₁₀₋₁₀₈ as analyzed by SDS-PAGE and visualized with Coomassie stain.

### DETAILED DESCRIPTION OF THE PREFERRED EMBODIMENTS

The present invention is predicated in part on the ability to discriminate between full length molecules and truncated or clipped variants during purification. This is particularly important for refolding of homomultimers such as homodimers. If truncated or non-full length molecules are co-purified with full length molecules, refolding can result in heteromultimers which may be inactive or exhibit undesirable properties.

Accordingly, one aspect of the present invention provides a method of purifying a peptide, polypeptide or protein from a biological sample said method comprising subjecting the biological sample to affinity chromatography comprising an affinity matrix under chromatographic conditions sufficient for the full length but not a truncated or non-full length peptide, polypeptide or protein corresponding to said fill length peptide, polypeptide or protein to be bound to or otherwise associate with the affinity matrix and then eluting said bound or associated peptide, polypeptide or protein from the affinity matrix and collecting same.

Generally, the peptide, polypeptide or protein is in recombinant form. Furthermore, the biological sample is generally a cell lysate, membrane preparation, cytoplasmic extract or other form containing inclusion bodies. The present invention extends, however, to biological samples in the form of culture supernatant fluid, fermentation fluid and conditioned medium.

Preferably, the affinity chromatography is conducted in a column in which case the chromatography is said to be conducted in an affinity chromatography column. The present invention extends to all other forms of chromatography. Reference herein to an affinity matrix includes reference to the solid support within the column or other apparatus to which the peptide, polypeptide or protein binds or otherwise associates. For example, if the affinity chromatography involves a metal chelate affinity chromatography column, a metal cation such as Ni⁺⁺ or Zn⁺⁺ is attached to or forms part of the affinity matrix.

The preferred chromatographic conditions are generally described as being “harsh” or “highly stringent” and these conditions enable full length peptide, polypeptide or protein to be bound or otherwise associated during affinity chromatography whereas truncated or “clipped” forms of the molecule are not retained and tend to wash through ahead of the full length molecule. The harsh chromatographic conditions include reducing conditions of from about 5-100 mM DTT for from about 10 minutes to about 4 hours. More preferred reducing conditions are from about 10-60 mM DTT for from about 20 minutes to about 3 hours.

The chromatographic conditions selected assist in reducing non-specific affinity binding to the chromatographic column. For example, in one preferred embodiment, the affinity chromatography is based on a binding or interacting property of an N-terminal or C-terminal region of the peptide, polypeptide or protein being purified.

Truncated or clipped forms of the peptide, polypeptide or protein are generally those molecules which substantially lack that region of the polypeptide which binds to or otherwise associates with the affinity column.

Accordingly, another aspect of the present invention is directed to a method of purifying a recombinant peptide, polypeptide or protein from a biological sample said method comprising subjecting said biological sample to affinity chromatography comprising an affinity matrix which has affinity for an N-terminal or C-terminal region of said peptide, polypeptide or protein but substantially not for the N-terminal or C-terminal region of a truncated or clipped form of said peptide, polypeptide or protein, said affinity chromatography being under chromatographic conditions sufficient to permit binding or association of full length but not truncated or non-full length peptide, polypeptide or protein, and then eluting the bound or associated peptide, polypeptide or protein from the affinity matrix and collecting same. Substantial affinity is not intended to include non-specific affinity.

In order to facilitate the purification process, an optional two-step affinity chromatography protocol is also contemplated by the present invention. For example, a first optional affinity chromatography may target an affinity region in one of the N-terminal or C-terminal portions of the peptide, polypeptide or protein. A second affinity chromatography step would then target the other of the N-terminal or C-terminal portions of the same molecule.

According to this embodiment, there is provided a method of purifying a peptide, polypeptide or protein from a biological sample comprising subjecting said biological sample to an optional first affinity chromatography comprising an affinity matrix which binds or associates said peptide, polypeptide or protein based on affinity to an N-terminal or C-terminal portion of said molecule, eluting off said bound or otherwise associated peptide, polypeptide or protein and subjecting same to a second affinity chromatography based on affinity to the other of an N-terminal or C-terminal portion of said molecule and eluting the peptide, polypeptide or protein bound or associated following said second affinity chromatography and collecting same.

Alternatively, cation exchange chromatography is used in place of a second affinity chromatography.

Accordingly, another aspect of the present invention provides a method of purifying a peptide, polypeptide or protein from a biological sample comprising subjecting said biological sample to an optional first affinity chromatography comprising an affinity matrix which binds or associates said peptide, polypeptide or protein based on affinity to an N-terminal or C-terminal portion of said molecule, eluting off said bound or otherwise associated peptide, polypeptide or protein and subjecting same to cation exchange chromatography and eluting the peptide, polypeptide or protein bound or associated following said cation exchange chromatography and collecting same.

In one embodiment, the first optional affinity chromatography step is based on an exogenous amino acid sequence fused to or otherwise associated with the N-terminal or C-terminal of said peptide, polypeptide or protein and the second affinity chromatographic step is based on an inherent feature of an amino acid sequence or structure of the N-terminus or C-terminus of the molecule.

In a particularly preferred example, the optional first affinity chromatographic step is based on a polymer of basic amino acids such as polyHis or hexa-His residues. Such residues have an affinity for metal cations such as a Ni⁺⁺ or Zn⁺⁺. The second affinity chromatographic step is, in a particularly useful example, based on an inherent heparin binding property of the peptide, polypeptide or protein.

On the basis of the highly charged putative heparin binding domain which exists in the COOH-terminus of VEGF-B₁₆₇, the charge of the truncated VEGF-B₁₆₇ species is expected to substantially different from the fall length form. A more preferred method would include the optional first affinity chromatographic step based on a polymer of basic amino acids such as polyHis or hexa-His residues, which have an affinity for metal cations such as a Ni⁺⁺ or Zn⁺⁺, followed by a second affinity chromatographic step based on the inherent charge difference in the C-terminal region of the fell length protein as compared to the truncated form. As stated above, cation exchange chromatography may be used to substitute for the second affinity chromatographic step.

The preferred peptide, polypeptide or protein of the present invention is a growth factor, cytokine or haemopoietic regulator of mammalian and preferably human origin. Reference to “mammalian” includes primates, humans, livestock animals, laboratory test animals and companion animals. A more preferred polypeptide or protein is a growth factor such as VEGF and in particular human-derived VEGF. A particularly preferred polypeptide or protein is VEGF-B or more particularly an isoform thereof such as VEGF-B₁₆₇, VEGF-B₁₈₆ or VEGF-B₁₀₋₁₀₈ (tagged or untagged with an amino acid sequence such as His₆). The amino acid sequence of VEGF-B₁₆₇ is shown in FIG. 1. The peptide, polypeptide or protein of the present invention is hereinafter exemplified in terms of a “VEGF-B isoform”. Reference hereinafter to “VEGF-B isoform” includes reference to VEGF-B and its derivatives and homologues and, in a preferred embodiment, refers to a human VEGF-B isoform. Derivatives of VEGF-B includes parts, portions, fragments, hybrid forms as well as single or multiple amino acid substitutions, deletions and/or additions as well as isoforms thereof such as VEGF-B₁₆₇, VEGF-B₁₈₆ and VEGF-B₁₀₋₁₀₈ as well as tagged forms thereof such as His₆ tagged VEGF-B₁₈₆ and His₆ tagged VEGF-B₁₀₋₁₀₈.

In a preferred embodiment, the VEGF-B isoform comprises a hexa-His at its N-terminal amino acid end portion and exhibits inherent heparin binding properties at its C-terminal amino acid end portion. This is referred to herein as a “tagged” VEGF-B isoform.

Accordingly, another aspect of the present invention contemplates a method of a purifying full length VEGF-B isoform or a related polypeptide from a biological sample, said method comprising subjecting said biological sample to a first optional affinity chromatography comprising an affinity matrix based on affinity binding to multiple contiguous exogenous His residues in the N-terminal portion of said VEGF-B isoform, eluting said VEGF-B isoform bound or otherwise associated with said first affinity chromatography and subjecting said eluted VEGF-B isoform to a second affinity chromatography based on affinity of the C-terminal portion of said VEGF-B isoform to heparin or like molecule, and then eluting and collecting said VEGF-B isoform bound or otherwise associated by said second affinity chromatography.

Generally, the second and optional first affinity chromatography are conducted under chromatographic conditions sufficient for the full length but not truncated or non-fall length VEGF-B isoform to be bound to or associated with the affinity chromatography.

In an alternative embodiment, cation exchange chromatography is used in place of the second affinity chromatographic step.

Accordingly, the present invention contemplates a method of purifying a full length VEGF-B isoform or a related polypeptide from a biological sample, said method comprising subjecting said biological sample to a first optional affinity chromatography comprising an affinity matrix based on affinity binding to multiple contiguous exogenous histidine (His) residues in the N-terminal portion of said VEGF-B isoform, eluting said VEGF-B isoform bound or otherwise associated with said first affinity chromatography and subjecting said eluted VEGF-B isoform to a cation exchange chromatography, and then eluting and collecting said VEGF-B isoform bound or otherwise associated by said cation exchange chromatography.

The collected, purified VEGF-B isoform or other polypeptide is generally subjected to refolding. The essence of this aspect of the present invention is that only full length monomers be available for refolding otherwise heteromultimers will result which may be inactive or exhibit undesirable properties. In a preferred embodiment, the peptide, polypeptide or protein and in particular the VEGF-B isoform is subjected to a cleavage reaction to remove any exogenous basic amino acids such as those introduced or otherwise associated with the N-terminal region.

Preferably, the purified monomeric forms of a VBGF-B isoform or other polypeptide are subjected to refolding conditions in 0.1-10 M GdCl, and more preferably 0.3-5 M GdCl followed by dialyzing against acetic acid or other suitable acid. Alternatively, arginine may be employed in the refolding conditions. The refolded multimeric polypeptides, and more preferably homomultimeric polypeptides are then subjected to purification by reversed phase chromatography or other convenient means.

Accordingly, in a particularly preferred embodiment, the present invention contemplates a method of purifying a homomultimeric polypeptide such as homodimeric VEGF-B₁₆₇ or similar molecule from a biological sample, said method comprising subjecting said biological sample to an optional first affinity chromatography based on affinity for exogenous basic amino acids such as polyHis or hexa-His in the N-terminal portion of said polypeptide; eluting and collecting fractions containing said polypeptide, subjecting said polypeptide to a second affinity chromatography based on affinity to heparin of the C-terminal portion of said polypeptide; eluting and collecting said polypeptide; subjecting said polypeptide to re folding conditions in the presence of GdCl or arginine and dialyzing the refolded polypeptide against acetic acid and/or other acid with similar properties; and purifying said refolded polypeptide by reversed phase chromatography.

In an alternative embodiment, the present invention provides a method of purifying a homomultimeric polypeptide such as a homodimeric VEGF-B isoform or similar molecule from a biological sample, said method comprising subjecting said biological sample to an optional first affinity chromatography based on affinity for exogenous basic amino acids such as polyHis or hexa-His in the N-terminal portion of said polypeptide; eluting and collecting fractions containing said polypeptide, subjecting said polypeptide to cation exchange chromatography, eluting and collecting said polypeptide; subjecting said polypeptide to refolding conditions in the presence of GdCl or arginine and dialyzing the refolded polypeptide against acetic acid and/or other acid with similar properties; and purifying said refolded polypeptide by reversed phase chromatography.

In a preferred aspects of the abovementioned embodiments, the refolded polypeptide is subjected to cleavage conditions to remove some or all of the exogenous basic amino acids such as polyHis or hexa-His prior to purification.

The present invention further contemplates compositions comprising purified peptide, polypeptide or protein prepared by the method of the present invention such a composition comprising purified homomultimeric forms of said peptide, polypeptide or protein. Preferred compositions comprise purified homodimeric forms of VEGF-B isoform or related molecule. The composition may also contain one or more pharmaceutically acceptable carriers and/or diluents.

Still another aspect of the present invention provides a method for the preparation and purification of a recombinant peptide, polypeptide or protein in homomultimeric form, said method comprising culturing a microorganism or animal cell line comprising a genetic sequence encoding a monomeric form of said peptide, polypeptide or protein under conditions sufficient for expression of said genetic sequence; obtaining cell lysate, culture supernatant fluid, fermentation fluid or conditioned medium from said microorganism or animal cell line and subjecting same to a first optional affinity chromatography step based on affinity to exogenous amino acids present in the N- or C-terminal region of said peptide, polypeptide or protein, collecting fractions containing said peptide, polypeptide or protein and subjecting said fractions to a second affinity chromatography step based on affinity to an inherent property of the amino acid sequence or structure in the C-terminal portion of said polypeptide such as binding to heparin or difference in charge; said affinity chromatography being under chromatographic conditions sufficient for fill length but not truncated or non-full length peptide, polypeptide or protein to be bound or otherwise associated by said affinity chromatography; eluting and collecting said full length peptide, polypeptide or protein and subjecting same to refolding conditions in the presence of GdCl or arginine and dialysing against acetic acid or other similar acid and then purifying the refolded polypeptide by reversed phase chromatography.

The present invention is further described by the following non-limiting Examples.

### EXAMPLE 1

### His₆-Tagged hman VEGF-B₁₆₇ Expression Vector

pET15b-VEGF-B₁₆₇

The coding region of the mature human VEGF-B₁₆₇ protein was amplified using PCR (94° C./2 min—1 cycle; 94° C./15 sec, 60° C./15 sec, 72° C./2 min—35 cycles; 72° C./5 min B 1 cycle; Stratagene pfu turbo; Corbett Research PC-960-G thermal cycler) to introduce in frame Nde I and BamH1 restriction enzyme sites at the 5′ and 3′ ends, respectively, using the following oligonucleotides:

| | | |
|---|---|---|
| 5′ Oligo | 5′-ATATCATATGGCCCCTGTCTCCCAGCCTGATGC-3′ | [<400>1] |
| 3′ Oligo | 5′-TATAGGATCCTCACCTTCGCAGCTTCCGCACCT-3′ | [<400>2] |

The resulting PCR derived DNA fragment was gel purified, digested with Nde I and BamH1, gel purified again, and then cloned into NdeI/BamH1 digested pET15b (Novagen, Madison Wis., USA). When expressed in E. coli the VEGF-B₁₆₇ protein has an additional 21 amino acids at the N-terminus that incorporates a hexa-His tag and a thrombin cleavage site (FIG. 1).

### EXAMPLE 2

### Expression of His₆-Tagged VEGF-B₁₆₇ in BL21(DE3) GOLD E. coli Cells Using pET15b-VEGF-B₁₆₇

pET15b-VEGF-B₁₆₇ was transformed into BL21(DE3) GOLD E. coli (Stratagene, Catalogue #230132) using an Electroporator (BioRad, USA) according to the manufacturer's instructions. The transformation reaction was plated onto LB ampicillin plates and incubated overnight at 37° C. Four ampicillin resistant colonies were picked, grown overnight and DNA extracted using a standard miniprep protocol (Bio101). Miniprep DNA was analyzed using the restriction enzymes BamH1 and Nde1. A colony giving the appropriate fragment was used for preparation of a glycerol stock for subsequent studies.

For preparation of a seed culture a 50 ml LB broth (10 g tryptone, 5 g yeast extract, 5 g NaCl, pH 7.0) was inoculated with pET15b-VEGF-B₁₆₇ transformed BL21(DE3) GOLD from the glycerol stock. The culture was allowed to grow at 37° C. (with continuous shaking) to OD₆₀₀ 0.7 and stored at 4° C. until required (usually no more than 4 days).

For protein production one litre of LB medium was inoculated with 5 ml of seed culture and incubated at 37° C. Cells were grown to OD₆₀₀ 0.7 (typically 5 hrs) and induced with 1 mM IPTG (Amersham Pharmacia, Sweden) for two hrs. Yields were typically 3-4 g wet cells per litre of culture (FIG. 2). Cells were pelleted by centrifugation and pellets stored frozen at −80° C. until required.

### EXAMPLE 3

### Isolation of His₆-Tagged VEGF-B₁₆₇ Inclusion Bodies

Cell Lysis

Frozen cell pellets were thawed and 3 ml lysis buffer (50 mM Tris-HCl, pH 8.0, 1 mM EDTA, 100 mM NaCl) was added per gram of cells. Once thoroughly mixed, 40 μl PMSF (10 mM) (phenylmethylsulfonyl fluoride: Sigma-Aldrich, USA) and 40 μl lysozyme (20 mg/ml) were added per gram of cells. The solution was mixed thoroughly and allowed to stand for 30 min at 37° C. Deoxycholic acid (4 mg/gram cells) was added and the solution mixed until viscous. DNase I (1 mg/ml: 20 μl/g of cells) was mixed with the cell lysate and allowed to stand for 30 min at 37° C., or until no longer viscous. Insoluble material (including inclusion bodies) was pelleted by centrifugation at 13,500 rpm for 30 min at 4° C. (FIG. 2).

Washing of Inclusion Bodies

Pelleted insoluble material was resuspended in 35 ml of 100 mM Tris-HCl, pH 7.0, 5 mM EDTA, 10 mM DTT, 2 M urea, 2% v/v Triton-X100 (Buffer 1) per litre of starting fermentation product. The suspension was placed on ice and subjected to sonication (6×1 min on high power with 2 min intervals) using a Braun sonicator, followed by centrifugation (13,500 rpm, 4° C.) for 30 min. This wash method was repeated two additional times. After the third wash, the pelleted material was resuspended in 25 ml of 100 mM Tris-HCl, pH 7.0, 5 mM EDTA, 10 mM DTT (Buffer 2) per litre of starting fermentation product, sonicated for one min at 4° C. and centrifuged (13,500 rpm, 4° C.) for 30 min. This second wash step was also repeated twice (FIG. 2). The washed inclusion bodies were pelleted as above and stored at −70° C. until required.

Solubilization

The washed inclusion bodies were solubilized by the addition of 10 ml 6M GdCl, 0.1 M NaH₂PO₄, 10 mM Tris-HCl, pH 8.5 (Buffer 3). In order to fully solubilize inclusion bodies, the suspension was placed on ice and subjected to sonication for one minute at high power. The solution was centrifuged at 18,000 rpm for 15 min in order to separate undissolved material. The solution was reduced by the addition of 20 mM DTT and allowed to stand at 37° C. for 30 min.

### EXAMPLE 4

### Purification of His₆-Tagged VEGF-B₁₆₇ from Isolated Inclusion Bodies

Ni²⁺ Affinity Chromatography

10 ml metal chelating resin was packed in a BioRad EconoPak column using Chelating Sepharose Fast Flow resin (Amersham Pharmacia, Sweden). The column was washed with three column volumes milliQ H₂O, followed by five column volumes of 0.1 M NiSO₄. A further three column volumes of milliQ H₂O followed by three column volumes of 6. M GdCl, 0.1 M NaH₂PO₄, 10 mM Tris-HCl, pH 8.5 (Buffer 3) were used to equilibrate the column. The reduced protein solution was loaded onto the column at 3 ml/min using a Pharmacia P1 peristaltic pump. To enhance recovery, the flow through was reapplied to the column five times prior to washing the column with three column volumes of the same buffer. The column was then washed with 5 column volumes of 8 M urea, 0.1 M NaH₂PO₄, 10 mM Tris-HCl, pH 8.5 (Buffer 4), followed by 5 column volumes of 8 M urea, 0.1 M NaH₂PO₄, 10 mM Tris-HCl, pH 6.3 (Buffer 5). The bound fraction was eluted with 6-10×5 ml volumes of 8 M urea, 0.1 M NaH₂PO₄, 10 mm Tris-HCl, 0.5 M Imidazole, pH 5.9. (Buffer 6). Fractions containing protein were identified by Bradford assay and an aliquot of each fraction was subjected to ethanol precipitation to remove the high salt content for subsequent analysis by SDS-PAGE electrophoresis. Samples were electrophoresed on an SDS-PAGE gel under reducing conditions. Coomassie staining revealed the major band to be running with an apparent molecular weight of 22 kDa FIG. 3A, lanes 1-5). To confirm its identity as VEGF-B₁₆₇, an identical gel was subjected to Western blot analysis using a polyclonal N-terminal VEGF-B peptide specific antibody.

Subsequent autoradiography indicated that this band was indeed VEGF-B₁₆₇ with additional bands corresponding to clipped forms of VEGF-B₁₆₇ also being observed (FIG. 3B, lanes 1 and 5). Total eluted protein was estimated to be approximately 30 mg by Bradford assay.

A second major band runs with an apparent molecular weight of approximately 18 kDa on SDS-PAGE under reducing conditions. Failure to remove this clipped variant would result in heterogenous forms of VEGF-B after refolding. Consequently, it was essential to develop a technique to remove the clipped form from the full-length VEGF-B₁₆₇ altogether. The use of heparin-sepharose under both reducing and denaturing conditions was successful in achieving this objective. It is likely that the clipped form does not possess the same charge profile as the putative C-terminal heparin-binding domain present on full-length VEGF-B₁₆₇.

Heparin Sepharose Affinity: Removal of C-Terminally Clipped VEGF-B

The pooled fractions from Ni²⁺ purification were reduced with 40 mM DTT for 1-2 hrs. A 10 ml heparin-sepharose CL6B column was prepared by first washing with S column volumes of milliQ H₂O and equilibrating with 4 column volumes of 6 M urea, 0.1 M NaH₂PO₄, 10 mM Tris-HCl, 1 mM EDTA, 20 mM DTT, pH 8.5 (Buffer 7). The urea concentration of the protein solution was reduced from 8 M to 6 M with 0.1 M NaH₂PO₄, 10 mM Tris-HCl, 1 mM EDTA, 20 mM DTT, pH 8.5. The protein solution was loaded onto the column at 3 ml/min. The C-terminally clipped VEGF-B eluted in the flow through and wash (FIGS. 3A and B, lane 6-7), while the full-length VEGF-B₁₆₇ eluted mainly with the addition of 6 M urea, 0.1 M NaH₂PO₄, 10 mM Tris-HCl, 1 mM EDTA, 20 mM DTT, 1 M NaCl, pH 8.5 (FIGS. 3A and B, lane 8). Total protein eluted was estimated to be approximately 18 mg by Bradford assay.

An Alternative Approach for the Removal of C-Terminally Clipped VEGF-B: Cation Exchange Chromatography

Pooled fractions from Ni²⁺ purification were reduced with 40 mM DTT for 1-2 hours. A 50 mL SP-Sepharose fast flow column (Amersham Pharmacia, Sweden) was prepared by equilibrating with five column volumes of 6 M urea, 10 mM NaH₂PO₄, 10 mM Tris-HCl, pH 5.8 Buffer 9). The protein solution was diluted three-fold with Buffer 9, and loaded onto the column at 10 mL/min. Full length monomeric VEGF-B₁₆₇ was separated from the truncated form using a linear gradient formed between buffer A and 6 M urea, 10 mM NaH₂PO₄, 10 mM Tris-HCl, 1M NaCl, pH 5.8 (Buffer 10) (see FIG. 13).

### EXAMPLE 5

### Refolding of Denatured Monomeric VEGF-B₁₆₇

1. Incorporation of GdCl in Refolding Buffer

Purified monomeric His₆-VEGF-B₁₆₇ from the heparin-sepharose purification was reduced with 20 mM DTT for 45 minutes at 37° C., followed by dilution to 60-200 μg/ml with Buffer 7 (6 M urea, 0.1 M NaH₂PO₄, 10 mM Tris-HCl, 1 mM EDTA, 20 mM DTT, pH 8.5). The protein solution was dialyzed at room temperature against Buffer 11 (100 mM Tris-HCl, 5 mM cysteine, 1 mM cystine, 0.5 M GdCl, pH 8.5) for one to three days.

2. Incorporation of Arginine in Refolding Buffer

Purified monomeric His₆-VEGF-B₁₆₇ from the heparin-sepharose purification was reduced with 20 mM DTT for 45 minutes at 37° C., followed by dilution to 60-200 μg/ml with Buffer 7 (6 M urea, 0.1 M NaH₂PO₄, 10 mM Tris-HCl, 1 mM EDTA, 20 mM DTT, pH 8.5). The protein solution was dialyzed at room temperature against Buffer 27 (100 mM Tris-HCl, 5 mM cysteine, 1 mM cystine, 0.4 M arginine, pH 8.5) for one to three days.

Major bands positioned at approximately 48 kDa and 22 kDa in Western blot analysis correspond to dimeric and monomeric forms of His₆-VEGF-B₁₆₇, respectively, under non-reducing conditions. In addition, higher oligomeric forms of His-VEGF-B₁₆₇ are present (FIG. 4). Coomassie staining suggested 20-40% conversion to dimer. The protein solution was dialyzed against 0.1 M acetic acid overnight and filtered through a 0.22 μM cellulose acetate filter (Corning, USA) to remove particulate matter.

### EXAMPLE 6

### Purification of Reformed Dimeric VEGF-B₁₆₇

The acidified protein solution was loaded onto a Brownlee C8 reversed-phase column pre-equilibrated at 45° C. in Buffer 12 (0.15% v/v Trifluoroacetic acid, TFA) using a Beckman GOLD liquid chromatographic system. Fractions were collected at one min intervals and monitored by SDS PAGE (FIG. 5) and Western blot analysis. A linear gradient was formed with Buffer 13 (0.13% v/v TFA, 60% v/v n-propanol; 0.5% v/v n-propanol/min). Fractions containing predominantly dimeric VEGF-B₁₆₇ were pooled, reapplied to the C8 column and eluted with a linear gradient formed between 20-45% of Buffer 13 (0.12% v/v n-propanol/min; FIG. 6). The purified dimeric VEGF-B₁₆₇ was reapplied to the C8 column and eluted with Buffer 13 to minimize sample dilution. Purified material was again analysed by SDS PAGE and Western blot analysis (FIG. 7). The purified VEGF-B₁₆₇ frequently appeared as two distinct bands running within 500 daltons of each other. This RP-HPLC purified VEGF-B₁₆₇ was subjected to N-terminal sequence analysis (Hewlett Packard, USA), resulting in 25 cycles of N-terminal sequence generating a single sequence with the expected N-terminus Ala-1. The sequence was consistent with the translated cDNA sequence of His₆-VEGF-B₁₆₇. Yields were approximately 1-2 mg/l of starting material.

### EXAMPLE 7

### An Alternative Method for the Purification of Refolded Dimeric VEGF-B₁₆₇

The acidified protein solution was loaded onto a Vydac 300 C8 reversed-phase column (2.2×10 cm; Higgins Analytical, USA) pre-equilibrated in Buffer 12 (0.15% v/v TFA) using a Beckman GOLD liquid chromatographic system. The column was washed with two column volumes of Buffer 12 followed by two column volumes of 35% Buffer 14 (60% v/v acetonitrile, 0.13% TFA). A linear gradient was formed with 35-60% Buffer 14 over 50 mins at a flow rate of 20 ml/min. Fractions containing dimeric His₆-VEGF-B₁₆₇ were pooled (as in Example 6), diluted ten-fold with Buffer 15 (80% v/v n-propanol, 10 mM NaCl, pH 2) and loaded on a Polyhydroxyethyl A hydrophilic column (2.1×25 cm; PolyLC, USA) pre-equilibrated with 25% Buffer 15. Dimeric protein was eluted using a linear gradient formed with 25-45% Buffer 16 (10 mM NaCl, pH 2.0). The purified dimeric His₆-VEGF-B₁₆₇ was diluted 10-fold with Buffer 12, reapplied to the C8 column and eluted with 100% v/v Buffer 14 to minimize sample dilution. Purified material was analysed by SDS PAGE and Western blot analysis (FIG. 8).

### EXAMPLE 8

### An Additional Alternative Method for the Purification of Refolded Dimeric His₆-VEGF-B₁₆₇

To separate dimeric His₆-VEGF-B₁₆₇ from mono- and multimeric species the acidified protein solution was diluted five-fold with Buffer 15 (80% v/v n-propanol, 10 mM NaCl, pH 2.0) and loaded onto a Polyhydroxyethyl A hydrophilic column (2.1×25 cm; PolyLC, USA) pre-equilibrated with three column volumes of Buffer 15 at 20 ml/min. The column was washed with two column volumes of 25% Buffer 16 (10 mM NaCl, pH 2.0). A linear gradient was formed with 25-45% Buffer 16 (10 mM NaCl, pH 2.0) over 40 minutes using a flow rate of 10 ml/min Fractions containing dimeric His₆-VEGF-B₁₆₇ were combined, diluted four-fold with Buffer 12 (0.15% TFA), and loaded onto a Vydac 300 C8 reversed-phase column (2.2×10 cm; Higgins Analytical, USA) pre-equilibrated with Buffer 12. The column was equilibrated with two column volumes of Buffer 12 followed by two column volumes of 35% Buffer 14 (60% v/v acetonitrile, 0.13% TFA). A linear gradient was formed with 3560% Buffer 14 over 50 mins at 20 ml/nun. Fractions containing dimeric His₆-VEGF-B₁₆₇ were pooled, diluted with Buffer 12, and reapplied to the C8 column. The protein was eluted with 100% Buffer 14 to minimize sample dilution.

### EXAMPLE 9

### Untagged Human VEGF-B₁₆₇ Expression Vector

Modified pET15b-VEGF-B₁₆₇

The coding region of the mature human VEGF-B₁₆₇ protein was amplified using PCR (96° C./2 min—1 cycle; 96° C./10 sec, 55° C./10 sec, 72° C./1 min—35 cycles; 72° C./2 min—1 cycle; Stratagene pfu turbo; Corbett Research PC-960-G thermal cycler) to introduce in frame Nco I and BamH1 restriction enzyme sites at the 5′ and 3′ ends, respectively, using the following oligonucleotides:

| | | |
|---|---|---|
| 5′Oligo | 5′-ATATCCATGGGCGGCCCCTGTCTCCCAGCCTGATGC -3′ | [<400>5] |
| 3′Oligo | 5′-TATAGGATCCTCACCTTCGCAGCTTCCGGCACCT -3′ | [<400>6] |

The resulting PCR derived DNA fragment was gel purified, digested with NcoI and BamH1, gel purified again, and then cloned into NcoI/BamH1 digested pET15b (Novagen, USA), resulting in the removal of the His₆-tag and thrombin cleavage site. When expressed in E. coli the untagged VEGF-B₁₆₇ protein has an additional glycine residue at the N-terminus.

### EXAMPLE 10

### Expression of Untagged VEGF-B₁₆₇ in BL21(DE3) GOLD E. coli Cells Using Modified pET15-VEGF-B₁₆₇

The modified pET15b-VEGF-B₁₆₇ was transformed into BL21(DE3) GOLD E. coli using an electroporator (BioRad, USA) according to the manufacturer's instructions. The transformation reaction was plated onto LB ampicillin plates and incubated overnight at 37° C. Sixteen ampicillin resistant colonies were picked, grown overnight and DNA extracted using a standard miniprep protocol (Bio101). Miniprep DNA was analyzed using the restriction enzymes BamH1 and NcoI. A colony giving the appropriate fragment was used for preparation of a glycerol stock for subsequent studies.

For preparation of a seed culture a 50 ml LB broth (10 g tryptone, 5 g yeast extract, 10 g NaCl, pH 7.5) was inoculated with pET15b-VEGF-B₁₆₇ transformed BL21(DE3) GOLD from the glycerol stock. The culture was allowed to grow at 37° C. (with continuous shaking) to OD₆₀₀ 0.7 and stored at 4° C. until required (usually no more than 4 days).

For protein production one litre of LB medium was inoculated with 20 ml of seed culture and incubated at 37° C. Cells were grown to OD₆₀₀ 0.7 (typically 3-4 hrs) and induced with 1 mM IPTG (Amersham Pharmacia Biotech, Sweden) for two hours. Yields were typically 3-4 g wet cells per litre of culture. Cells were pelleted by centrifugation and pellets stored frozen at −80° C. until required

### EXAMPLE 11

### Isolation of Untagged VEGF-B₁₆₇ Inclusion Bodies

Cell Lysis

Frozen cell pellets were thawed and 3 ml lysis buffer (50 mM Tris-HCl, pH 8.0, 1 mM EDTA, 100 mM NaCl) was added per gram of cells. Once thoroughly mixed, 40 μl PMSF (10 mM) and 40 μl lysozyme (20 mg/ml) were added per gram of cells. The solution was mixed thoroughly and allowed to stand for 1 hour at 37° C. Deoxycholic acid (4 mg/gram cells) was added and the solution mixed until viscous. DNase I (1 mg/ml: 20 μl/g of cells) was mixed with the cell lysate and allowed to stand for 30 min at 37° C., or until no longer viscous. Insoluble material (including inclusion bodies) was pelleted by centrifugation at 13,500 rpm for 45 min at 4° C.

Washing of Inclusion Bodies

Pelleted insoluble material was resuspended in 35 ml of Buffer I (100 mM Tris-HCl, pH 7.0, 5 mM EDTA, 10 mM DTT, 2 M urea, 2% v/v Triton X-100) per litre of starting fermentation product: The suspension was placed on ice and subjected to sonication (6×1 min on high power with 2 min intervals), followed by centrifugation (13,500 rpm, 4° C.) for 30 min. This wash method was repeated two additional times. After the third wash, the pelleted material was resuspended in 25 ml of Buffer 2 (100 mM Tris-HCl, pH 7.0, 5 mM EDTA, 10 mM DTT) per litre of starting fermentation product, sonicated for one min at 4° C. and centrifuged (13,500 rpm, 4° C.) for 30 min. This second wash step was also repeated twice. The washed inclusion bodies were pelleted as above and stored at −70° C. until required.

Solubilization

The washed inclusion bodies were solubilized by the addition of 20 ml Buffer 3 (6 M GdCl, 10 mM NaH₂PO₄, 10 mM Tris-HCl, pH 8.5). In order to fully solubilize inclusion bodies, the suspension was placed on ice and subjected to sonication for one minute at high power. The solution was centrifuged at 18,000 rpm for 15 min in order to separate undissolved material. The solution was reduced by the addition of 20 mM DTT, 1 mM EDTA and allowed to stand at 37° C. for 2 hours.

### EXAMPLE 12

### Purification of Untagged VEGF-B₁₆₇ from Isolated Inclusion Bodies

Cation Exchange Chromatography

A 50 ml SP-Sepharose column (Amersham Pharmacia Biotech, Sweden) was prepared by equilibrating the column with five column volumes of Buffer 9 (6 M urea, 10 mM NaH₂PO₄, 10 mM Tris-HCl, pH 5.8). The protein solution was adjusted to pH 5.8, and loaded onto the column at 5 ml/min. Full length monomeric VEGF-B₁₆₇ was separated from the truncated form and other contaminating host cell proteins using a linear gradient formed between Buffer 9 and Buffer 10 (6 M urea, 10 mM NaH₂PO₄, 10 mM Tris-HCl, 1M NaCl, pH 5.8).

### EXAMPLE 13

### Refolding of Denatured Monomeric Untagged VEGF-B₁₆₇

Purified monomeric untagged VEGF-B₁₆₇ from the cation exchange purification was reduced with 20 mM DTT for 45 minutes at 37° C., followed by dilution to 60-100 μg/ml with Buffer 7 (6 M urea, 0.1 M NaH₂PO₄, 10 mM Tris-HCl, 1 mM EDTA, 20 mM DTT, pH 9.5). The protein solution was dialyzed at room temperature against Buffer 11 (100 mM Tris-HCl, 5 mM cysteine, 1 mM cystine, 2 mM EDTA, 0.5 M GdCl, pH 8.5) for one to three days. Major bands positioned at approximately 48 kDa and 22 kDa in Western blot analysis correspond to dimeric and monomeric forms of untagged VEGF-B₁₆₇, respectively, under non-reducing conditions. In addition, higher oligomeric forms of untagged VEGF-B₁₆₇ are present.

The protein solution was dialyzed against 0.1 M acetic acid overnight and filtered through a 0.22 μM cellulose acetate filter (Corning, USA) to remove particulate matter.

### EXAMPLE 14

### Purification of Untagged Refolded Dimeric VEGF-B₁₆₇

To separate dimeric untagged VEGF-B₁₆₇ from mono- and multimeric species the acidified protein solution was diluted five-fold with Buffer 15 (80% v/v n-propanol 10 mM NaCl, pH 2.0) and loaded onto a Polyhydroxyethyl A hydrophilic column (2.1×25 cm; PolyLC, USA) pre-equilibrated with three column volumes of Buffer 15 at 20 ml/min. The column was washed with two column volumes of 25% Buffer 16 (10 mM NaCl, pH 2.0). A linear gradient was formed with 25-45% Buffer 16 over 40 minutes using a flow rate of 10 ml/min. Fractions containing dimeric VEGF-B₁₆₇ were combined, diluted four-fold with Buffer 12 (0-15% TFA), and loaded onto a Vydac 300 C8 reversed-phase column (2.2×10 cm; Higgins Analytical, USA) pre equilibrated with Buffer 12. The column was washed with two column volumes of Buffer 12 followed by two column volumes of 35% Buffer 14 (60% v/v acetonitrile, 0.13% TFA). A linear gradient was formed with 35-60% Buffer 14 over 50 mins at 20 ml/min- Fractions containing dimeric VEGF-B₁₆₇ were pooled, diluted with Buffer 12, and reapplied to the C8 column. The protein was eluted with 100% Buffer 14 to minimize sample dilution.

### EXAMPLE 15

### Human His₆-VEGF-B₁₈₆ Expression Vector

pET15b-VEGF-B₁₈₆

The coding region of the mature human VEGF-B₁₈₆ protein was amplified using PCR (94° C./2 min—1 cycle; 94° C./15 sec, 60° C./15 sec, 72° C./2 min—35 cycles; 72° C./5 min—1 cycle; Stratagene pfu turbo; Corbett Research PC-960-G thermal cycler) to introduce in frame Nde I and BamH1 restriction enzyme sites at the 5′ and 3′ ends, respectively, using the following oligonucleotides:

| | | |
|---|---|---|
| 5′Oligo | 5′-TATACATATGGCCCCTGTCTCCCAGCCTGATGC-3′ | [<400>7] |
| 3′Oligo | 5′-TATAGGATCCTTATCACCTTCGCAGCTTCCGGC-3′ | [<400>8] |

The resulting PCR derived DNA fragment was gel purified, digested with NdeI and BamH1, gel purified again, and then cloned into NdeI/BamH1 digested pET15b (Novagen, USA). When expressed in E. coli the VEGF-B₁₆₇ protein has an additional 21 amino acids at the N-terminus that incorporates a hexa-His tag and a thrombin cleavage site.

### EXAMPLE 16

### Expression of His₆-Tagged VEGF-B₁₈₆ in BL21(DE3) GOLD E. coli Cells Using pET15b-VEGF-B₁₈₆

The pET15VEGF-B₁₈₆ was transformed into BL21(DE3) GOLD E. coli using an electroporator (BioRad, USA) according to the manufacturer's instructions. The transformation reaction was plated onto LB ampicillin plates and incubated overnight at 37° C. Four ampicillin resistant colonies were picked, grown overnight and DNA extracted using a standard miniprep protocol (Bio101). Miniprep DNA was analyzed using the restriction enzymes BamH1 and Nde1 . A colony giving the appropriate fragment was used for preparation of a glycerol stock for subsequent studies. For preparation of a seed culture a 50 ml LB broth (10 g tryptone, 5 g yeast extract, 5 g NaCl, pH 7.0) was inoculated with pET15b-VEGF-B₁₈₆ transformed BL21(DE3) GOLD from the glycerol stock. The culture was allowed to grow at 37° C. (with continuous shaking) to OD₆₀₀ 0.7 and stored at 4° C. until required (usually no more than 4 days).

For protein production one litre of LB medium was inoculated with 5 ml of seed culture and incubated at 37° C. Cells were grown to OD₆₀₀ 0.7 (typically 5 hrs) and induced with 1 mM IPTG for two hrs. Yields were typically 3-4 g wet cells per litre of culture. Cells were pelleted by centrifugation and pellets stored frozen at −80° C. until required.

### EXAMPLE 17

### Isolation of His₆-Tagged VEGF-B₁₈₇ Inclusion Bodies

Cell Lysis

Frozen cell pellets were thawed and 20 ml lysis buffer (50 mM Tris-HCl, pH 7.5, 1 mM EDTA, 100 mM NaCl) was added per gram of cells. Once thoroughly mixed, 40 μl PMSF (10 mM) and 40 μl lysozyme (20 mg/ml) were added per gram of cells. The solution was mixed thoroughly and allowed to stand for 30 min at 37° C. Deoxycholic acid (4 mg/gram cells) was added and the solution mixed until viscous. DNase I (1 mg/ml: 20 μl/g of cells) was mixed with the cell lysate and allowed to stand for 30 min at 37° C., or until no longer viscous. Insoluble material (including inclusion bodies) was pelleted by centrifugation at 13,500 rpm for 30 min at 4° C.

Washing of Inclusion Bodies

Pelleted insoluble material was resuspended in 100 ml of Buffer 22 (50 mM Tris-HCl, pH 7.5, 1 mM EDTA, 100 mM NaCl) per litre of starting fermentation product. The suspension was placed on ice and subjected to sonication (6×1 min on high power with 2 min intervals), followed by centrifugation (13,500 rpm, 4° C.) for 30 min. The pelleted material was resuspended in 50 ml of Buffer 23 (2 M urea, 100 mM Tris-HCl, pH 7.5, 100 mM NaCl, 5 mM EDTA) per litre of starting fermentation material, sonicated for one min at 4° C. and centrifuged (13,500 rpm, 4° C.) for 30 min. This second wash step was repeated twice. The washed inclusion bodies were pelleted as above and stored at −70° C. until required.

Solubilization

The washed inclusion bodies (2.5 g) were solubilized by the addition of 1 L of Buffer 24 (8 M urea, 100 mM Tris-HCl, 50 mM NH₄SO₄, 5% (v/v) Triton X-100, 100 mM DTT, pH 9.0). In order to fully solubilize inclusion bodies, the suspension was homogenized with an Ultra-turrax T8 homogenizer (Janke & Kunkel GmbH, Germany) for 3 min at full power and then incubated at 45° C. for 1 hour.

### EXAMPLE 18

### Purification of His₆-Tagged VEGF-B₁₈₆ from Isolated Inclusion Bodies

Cation Exchange Chromatography

This method describes a means by which a truncated component of His₆-VEGF-B₁₈₆ may be selectively separated from full length His₆-VEGF-B₁₈₆. This shortened His₆-VEGF-B₁₈₆ component appears to non-covalently associate with the full-length material. This interaction can be disrupted by the presence of the non-ionic detergent Triton X-100.

The solubilized inclusion bodies suspension was adjusted to pH 5.8 prior to loading on a 100 ml SP-sepharose cation exchange column (Amersham Pharmacia Biotech, Sweden) pre-equilibrated with three column volumes of Buffer 25 (4 M urea, 100 mM Tris-HCl, 50 mM NH₄SO₄, 1% Triton X-100, 2.5 mM P-mercaptoethanol, pH 5.8). The sample was loaded through the system pump of an ÅKTA Explorer 100 (Amersham Pharmacia Biotech, Sweden) at a flow rate of 10 ml/min. Bound material was washed with 10 column volumes of Buffer 25. The bound material was eluted with a gradient generated over 5 column volumes from 0-100% Buffer 26 (4 M urea, 0.1 M Tris-HCl, 50 mM NH₄SO₄, 1% Triton X-100, 2.5 mM f-mercaptoethanol, 1 M NaCl, pH 5.8). Eluant was fractionated into 1 minute/10 ml fractions. Those fractions within the conductivity range of 15-75 mS/cm were pooled and diluted 10-fold with Buffer 24 (8M urea, 100 mM Tris-HCl, 50 mM NH₄SO₄, 5% v/v Triton X-100, 100 mM DTT pH 9.0). The solution was adjusted to pH 9.0 and incubated at 45° C. for 1 hr. The solution was readjusted to pH 5.8 and the previous chromatography step repeated. Collected fractions were analyzed by SDS-PAGE Coomassie and Western blot analysis using VEGF-B-specific monoclonal antibodies.

### EXAMPLE 19

### Refolding of Monomeric His₆-VEGF-B₁₈₆

The purified monomeric His₆-VEGF-B₁₈₆ was reduced with 20 mM DTT for 45 minutes at 37° C., followed by dilution to 60-200 μg/mL with Buffer 7 (6 M urea, 0.1 M NaH₂PO₄, 10 mM Tris-HCl, 1 mM EDTA. 20 mM DTT, pH 8.5). The protein solution was dialyzed at room temperature against Buffer 11 (100 mM Tris-HCl, 5 mM cysteine, 1 mM cystine, 0.5 M GdCl, pH 8.5) for one to three days. Major bands corresponding to dimeric and monomeric forms of His₆-VEGF-B₁₈₆ were identified in addition to higher oligomeric forms of His₆-VEGF-B₁₈₆₆. Coomassie staining suggested >20% conversion to dimer. The protein solution was dialyzed against 0.1 M acetic acid overnight and filtered through a 0.22 μM cellulose acetate filter (Corning, USA) to remove particulate matter.

### EXAMPLE 20

### Purification of Refolded Dimeric His₆-VEGF-B₁₈₆

To separate dimeric His₆-VEGF-B₁₈₆ from mono- and multimeric species the acidified protein solution was diluted five-fold with Buffer 15 (80% v/v n-propanol, 10 mM NaCl. pH 2.0) and loaded onto a Polyhydroxyethyl A hydrophilic column (2.1×25 cm; PolyLC, USA) pre-equilibrated with three column volumes of Buffer 15 at 20 ml/min. The column was washed with two column volumes of 25% Buffer 16 (10 mM NaCl, pH 2.0). A linear gradient was formed with 25-45% Buffer 16 (10 mM NaCl, pH 2.0) over 40 minutes at a flow rate of 10 ml/min. Fractions containing dimeric His₆-VEGF-B₁₈₆ were combined, diluted four-fold with Buffer 12 (0.15% TFA), and loaded onto a Vydac 300 C8 reversed-phase column (2.2×10 cm; Higgins Analytical, USA) pre-equilibrated with Buffer 12. The column was washed with two column volumes of Buffer 12 followed by two column volumes of 35% Buffer 14 (60% v/v acetonitrile, 0.13% TFA). A linear gradient was formed with 35-60% Buffer 14 over 50 nuns at 20 ml/min. Fractions containing dimeric His₆-VEGF-B₁₈₆ were pooled, diluted with Buffer 12, and reapplied to the C8 column. The purified dimeric protein was eluted with 100% Buffer 14 to minimize sample dilution (FIG. 14).

### EXAMPLE 21

### Human VEGF-B₁₀₋₁₀₈ Expression Vector

pQE30-VEGF-B₁₀₋₁₀₈

The coding region of the mature human VEGF-B₁₀₋₁₀₈ protein was amplified using PCR (95° 2 min—1 cycle; 94° C./1 min, 60° C./1 min, 72° C./1 min—30 cycles; 72° C./15 min—1 cycle; 1.5 U Expand High Fidelity PCR System enzyme mix (Roche Diagnostics GrmbH, Germany; Corbett Research PC-960-G thermal cycler) to introduce in frame BamHI and HindIII restriction enzyme sites at the 5′ and 3′ ends, respectively, using the following oligonucleotides:

| | | |
|---|---|---|
| 5′Oligo | 5′- CACGGATCCGCAGCACACTATCACCAGAGGAAAG -3′ | [<400>9] |
| 3′Oligo | 5′- GCATAAGCTTTCACTTTTTTTTAGGTCTGCATTC -3′ | [<400>10] |

The resulting PCR derived DNA fragment was gel purified, digested with BamHI and HindIII, gel purified again, then cloned into BamHI and HindIII digested pQE30 (QIAGEN GmbH, Germany). The ligated DNA was transformed into DH5α E. coli using an electroporator (BioRad, USA) according to the manufacturer's instructions. The transformation reaction was plated onto LB ampicillin plates and incubated overnight at 37° C. Six ampicillin resistant colonies were picked for colony PCR analysis using pQE30 primers (QIAGEN GmbH, Germany) to identify fragment insertion. Colonies with the appropriate fragment were grown overnight and the plasmid DNA extracted using a standard miniprep protocol (QIAGEN GmbH, Germany). The DNA was sequenced using a BigDye Sequencing Kit (Applied Biosystems, USA). When expressed in E. coli the VEGF-B₁₀₋₁₀₈ protein has an additional 16 amino acids at the N-terminus that incorporates a hexa-His tag and a Genenase I (New England Biolabs, USA) cleavage site.

### EXAMPLE 22

### Expression of His₆-Tagged VEGF-B₁₀₋₁₀₈ in M15[pREP4] E. coli Cells Using pQE30-VEGF-B₁₀₋₁₀₈

The pQE30-VEGF-B₁₀₋₁₀₈ was transformed into M15[pREP4] E. coli (QIAGEN GmbH, Germany) using an electroporator (BioRad, USA) according to the manufacturer's instructions. The transformation reaction was plated onto LB ampicillin and kanamycin plates and incubated overnight at 37° C. A single ampicillin and kanamycin resistant colony was picked, grown overnight and used for preparation of a glycerol stock for subsequent studies.

For preparation of a seed culture a 50 ml LB broth (10 g tryptone, 5 g yeast extract, 5 g NaCl, pH 7.0) with ampicillin and kanamycin was inoculated with pQE30-VEGF-B₁₀₋₁₀₈ transformed M15[pREP4] from the glycerol stock The culture was allowed to grow overnight at 37° C. with continuous shaking.

For protein production one litre of LB medium with ampicillin and kanamycin was inoculated with 20 ml of seed culture and incubated at 37° C. Cells were grown to OD₆₀₀ 0.7 (typically 4 hrs) and induced with 1 mM IPTG (Amersham Pharmacia Biotech, Sweden) for 4 hrs. Yields were typically 5-6 g wet cells per litre of culture. Cells were pelleted by centrifugation and pellets stored frozen at −80° C. until required

### EXAMPLE 23

### Isolation of His₆-Tagged VEGF-B₁₀₋₁₀₈ Inclusion Bodies

Cell Lysis

Frozen cell pellets were thawed and 3 ml lysis buffer (50 mM Tris-HCl, pH 8.0, 1 mM EDTA, 100 mM NaCl) was added per gram of cells. Once thoroughly mixed, PMSF (40 μl, 10 mM) and lysozyme (40 μl, 20 mg/ml) were added per gram of cells. The solution was mixed thoroughly and allowed to stand for 30 min at 37° C. Deoxycholic acid (4 mg/gram cells) was added and the solution mixed until viscous. DNase I (1 mg/ml: 20 μl/g of cells) was mixed with the cell lysate and allowed to stand for 30 min at 37° C., or until no longer viscous. Insoluble material (including inclusion bodies) was pelleted by centrifugation at 13,500 rpm for 30 min at 4° C.

Washing of Inclusion Bodies

Pelleted insoluble material was resuspended in 35 ml of Buffer 1 (100 mM Tris-HCl, pH 7.0, 5 mM EDTA, 10 mM DTT, 2 M urea, 2% v/v Triton X-100) per litre of starting fermentation product. The suspension was placed on ice and subjected to sonication (6×1 min on high power with 2 min intervals; Braun, Germany), followed by centrifugation (13,500 rpm, 4° C.) for 30 min. This wash method was repeated two additional times. After the third wash, the pelleted material was resuspended in 25 ml of Buffer 2 (100 mM Tris-HCl, pH 7.0, 5 mM EDTA, 10 mM DTT) per litre of starting fermentation product, sonicated for one min at 4° C. and centrifuged (13,500 rpm, 4° C.) for 30 min. This second wash step was also repeated twice. The washed inclusion bodies were pelleted as above and stored at −70° C. until required.

### EXAMPLE 24

### Purification of His₆-VEGF-B₁₀₋₁₀₈ from Isolated Inclusion Bodies

Solubilization

The washed inclusion bodies were solubilized by the addition of 20 ml Buffer 3 (6M GdCl, 0.1 M NaH₂PO₄, 10 mM Tris-HCl, pH 8.5). In order to fully solubilize inclusion bodies, the suspension was placed on ice and subjected to sonication for one minute at high power. The solution was reduced by the addition of 20 mM P-mercaptoethanol and incubated at 37° C. for 30 min. Insoluble material was removed by centrifugation at 18,000 rpm for 15 min.

Ni²⁺ Affinity Chromatography

A column containing 20 ml Ni-NTA Superflow resin (QIAGEN GmbH, Germany) was washed with 10 column volumes of milliQ H₂O followed by five column volumes of Buffer 3. The reduced protein solution was loaded onto the column at 4 ml/min and washed with five volumes of Buffer 3. The bound non-specific endogenous bacterial proteins were removed from the column by washing with five column volumes of Buffer 17 (6M GdCl, 0.1 M NaH₂PO₄, 10 mM Tris-HCl, 20 mM imidazole, pH 6.3) followed by five column volumes of Buffer 3. The bound protein was eluted with 10 column volumes of Buffer 18 (6M GdCl, 0.1 M NaH₂PO₄, 10 mM Tris-HCl, pH 4.5). The fractions containing His₆-tagged VEGF-B₁₀₋₁₀₈, as determined by Western blot analysis using a polyclonal N-terminal VEGF-B peptide specific antibody and corresponding to the single peak on the elution profile, were pooled and stored at 4° C.

### EXAMPLE 25

### Refolding of Denatured Monomeric VEGF-B₁₀₋₁₀₈

The purified monomeric His₆-VEGF-B₁₀₋₁₀₈ was adjusted to pH 8.5 with 5 M NaOH and reduced with 20 mM DTT for 2 hrs at 37° C. The protein solution was diluted 10-fold by the slow dropwise addition of Buffer 11 (100 mM Tris-HCl pH 8.5, 5 mM cysteine, 1 mM cystine, 0.5 M GdCl, 2 mM EDTA, pH 8.5) at 4° C. followed by overnight dialysis against 0.1 M acetic acid. Major bands positioned at approximately 13 kDa and 26 kDa in Western blot analysis correspond to monomeric and dimeric forms of His₆-VEGF-B₁₀₋₁₀₈, respectively, under non-reducing conditions. Coomassie staining suggested 30-40% conversion to dinner.

### EXAMPLE 26

### Purification of Refolded Dimeric His₆-VEGF-B₁₀₋₁₀₈

The acidified protein solution was concentrated five-fold with a 10 kDa cut-off EasyFlow concentrator (Sartorius AG, Germany), and adjusted to contain 80% n-propanol, 10 mM NaCl, pH 2.0. The material was loaded onto a Polyhydroxyethyl A hydrophilic column (2.1×25 cm; PolyLC, USA) attached to an ÅKTA FPLC system (Amersham Pharmacia Biotech, Sweden) at 10 ml/min, and equilibrated with Buffer 15 (80% n-propanol, 10 mM NaCl, pH 2.0). The bound material was eluted with a 10-40% linear gradient over 60 min of Buffer 16 (10 mM NaCl, pH 2.0).

Fractions containing dimeric His₆-VEGF-B₁₀₋₁₀₈ were pooled and diluted five-fold with Buffer 12 (0.15% v/v TFA). The material was loaded onto a Vydac 300 C8 Reverse-phase column (2.2×10 cm; Higgins Analytical, USA) previously equilibrated with Buffer 12 (0.15% v/v TFA) at 10 ml/min. The bound material was eluted with a 50-65% linear gradient over 60 min of Buffer 14. (0.13% v/v TFA, 60% v/v acetonitrile). Fractions containing dimeric VEGF-B₁₀₋₁₀₈ were pooled, diluted five-fold in Buffer 12 and reloaded on to the C8 column equilibrated with Buffer 12. Purified dimeric His₆-VEGF-B₁₀₋₁₀₈ was eluted with 100% Buffer 14 and freeze dried (FIG. 15). Yields were approximately 16 mg/ of starting culture.

### EXAMPLE 27

### Purified Dimeric VEGF-B₁₆₇ Binds VEGF Receptor R1 (VEGF-R1/Flt-1)

Members of the VEGF family of cytokines have been shown to bind differentially to a family of three receptor tyrosine kinases (RTKs) designated VEGF receptor 1 (VEGF-R1), 2 (VEGF-R2) and 3 (VEGF-R3). Demonstration of binding to one or more of these receptors is important to establish that the purified homodimer has refolded correctly. The inventors used two methods, biosensor analysis (surface plasmon resonance) and an ELISA based assay, to demonstrate that the refolded dimeric VEGF-B₁₆₇ is able to bind to VEGF-R1

Biosensor Analysis of Receptor Binding

Analysis of binding of VEGF-B₁₆₇ to VEGF-R1 and VEGF-R2 was monitored using surface plasmon resonance (Biacore 2000, Pharmacia-Biosensor, Sweden) and commercially available receptor proteins. For control purposes binding of the receptors to VEGF-A₁₆₅ was also monitored. Both VEGF-B₁₆₇ and VEGF-A₁₆₅ were individually immobilised to a sensorchip using NHS/EDC chemistry according to the manufacturer's instructions. Briefly, 35 μl of NHS and EDC was injected onto the sensorchip at a flow rate of 5 μl/min to activate the sensor surface and enable covalent coupling of either VEGF-A₁₆₅ or VEGF-B₁₆₇. The VEGF-A₁₆₅ (Peprotech, USA, 100 μg/ml) was diluted (1:10) in 20 mM sodium acetate, pH 4.2 and injected directly onto the sensor surface (35 μl). Post coupling, diaminoethane (50 mM, pH 9.0) was used to block any unbound activated sites on the sensor surface. Concentrated dimeric VEGF-B₁₆₇ (200 μg/ml) was diluted (1:10) in 20 mM sodium acetate and immobilized onto a separate channel on the sensorchip. Post coupling, diaminoethane (50 mM, pH 9.0) was used to block any unbound activated sites on the sensor surface.

At the end of each run, the surface of the sensorchip was regenerated using 2 cycles of phosphoric acid (0.1 M, 30 μl) at a flow of 50 μl/min. Both VEGF-R1 (R&D systems, USA) and VEGF-R2 (R&D systems, USA) were obtained as chimeric proteins incorporating the human immunoglobulin Fc domain. Both were diluted into 0.1% w/v BSA in PBS as a stock solution (50 μg/ml, storage −20° C.).

Biosensor analysis of binding of VEGF-A₁₆₅ or VEGF-B₁₆₇ to VEGF-R2/Fc is shown in FIG. 9A. VEGF-R2/Fc was diluted 1:10 in Buffer 19 (20 mM HEPES, 0.15 M NaCl, 0.005% v/v Tween20, 3.4 mM EDTA, pH 7.4) and subsequently run over both VEGF-A₁₆₅ and VEGF-B₁₆₇ channels simultaneously. VEGF-R2/Fc bound specifically to VEGF-A₁₆₅ (933 RU's) but not to VEGF-B₁₆₇ (2 RU's). Biosensor analysis of binding of VEGF-A₁₆₅ or VEGF-B₁₆₇ to VEGF-R1/Fc is shown in FIG. 9B. In contrast to VEGF-R2/Fc, VEGF-R1/Fc bound to both VEGF-A₁₆₅ (1764 RtJ's) and VEGF-B₁₆₇ (1323 RU's).

ELISA Based Analysis of Receptor Binding

An ELISA based assay to facilitate competitive receptor binding studies was developed using the chimeric receptor proteins described above and, in addition, a biotinylated polyclonal antibody specific for VEGF-A₁₆₅. In the first instance, surface plasmon resonance was used to verify the specificity of the antibody. Binding to sensorchip immobilised (see above) VEGF-A₁₆₅ and VEGF-B₁₆₇ is shown in FIG. 10. In this example, the biotinylated anti-VEGF-A₁₆₅ antibody (R&D systems, USA) bound specifically to VEGF-A₁₆₅ (790 RU's) but not to VEGF-B₁₆₇ (0.4 RU's). For control purposes, the inventors also examined the binding of an affinity purified rabbit VEGF-B specific polyclonal antibody to VEGF-A₁₆₅ and VEGF-B₁₆₇. This antibody bound specifically to VEGF-B₁₆₇ (313 RU's) but not to VEGF-A₁₆₅ (1.4 RU's).

The potential of VEGF-B₁₆₇ to compete with VEGF-A₁₆₅ for binding to VEGF-R1 was examined in an ELISA based assay using the VEGF-R1/Fc chimeric receptor. Briefly, the assay utilised the following protocol:

1. 100 μl of rabbit anti-human IgG (Silenus, Australia, 8 μg/ml in PBS) was added to each well of a 96 well plate (Nunc, Maxisorp), and incubated overnight at 4° C.

2. Plates were washed three times with Buffer 20 (PBS, 0.1% v/v BSA, 0.05% v/v Tween 20) then blocked with 300 μl/well of Buffer 21 (1% w/v BSA, 5% w/v sucrose 0.05% w/v sodium azide for 1 hr at room temp.

3. Plates were washed as above and then 100 μl of VEGF-R1/Fc (100 ng/ml in Buffer 20) added. Plates were incubated for 90 min at room temperature.

4. Wash plates as in step 2.

5. VEGF-A₁₆₅ was added (indicated concentration in Buffer 20) and incubated at room temp for 1 hr. In competition experiments, VEGF-B₁₆₇ was added 30 min prior to the addition of VEGF-A₁₆₅. A range of VEGF-B₁₆₇ concentrations were used to compete with VEGF-A₁₆₅ at a final concentration of 10 ng/ml.

6. Wash plates as in step 2.

7. Biotinylated anti-VEGF-A₁₆₅ (10 ng/ml in Buffer 20, 100 μl) was added and incubated for 1 hr.

8. Wash plates as in step 2.

9. Binding of VEGF-A₁₆₅ antibody was detected by addition of 100 μl of a 1:10,000 dilution of streptavidin-horseradish peroxidase (SA-HRPO; Sigma, 1.0 mg/ml) followed by incubation at room temp for 30 min.

10. Wash plates as in step 2.

11. Complex formation was detected by addition of 100 μl/well of tetramethylbenzidine (TMB) substrate solution (Silenus, Australia) to each well. After addition of 50 μl of stop solution (0.5 M H₂SO₄) optical density was measured at 450 nm.

FIG. 11 shows the binding of VEGF-A₁₆₅ (1 pg-1 μg) to both VEGF-R1 and VEGF-R2 using a range of receptor concentrations (10 ng/ml-100 ng/ml). No significant non-specific binding was detected in control samples. In this example, binding of VEGF-A₁₆₅ to each receptor was directly proportional to both VEGF-A₁₆₅ and receptor concentrations. VEGF-B₁₆₇ was able to compete with VEGF-A₁₆₅ for binding to VEGF-R1 as shown in FIG. 12. VEGF-B₁₆₇ inhibited 50% of the VEGF-A₁₆₅ (10 ng/ml) binding at a concentration of approximately 20 ng/ml in this assay.

Receptor binding data obtained using Biosensor and ELISA based analysis clearly indicate that the production, refolding and purification protocol gives rise to VEGF-B₁₆₇ that is refolded into the conformation capable of binding to the receptor. In addition the competitive binding analysis suggests that the majority of purified dimer is active, consistent with appropriately folded conformation.

### EXAMPLE 28

### A Novel Bioassay Based on Chimeric Receptors Demonstrates that Refolded VEGF-B Isoforms are Biologically Active

Naturally occurring VEGF-B isoforms (VEGF-B_{167 and 186}) as well as artificial truncated versions of the protein (VEGF-B₁₀₋₁₀₈) that retain the core structural domain bind to VEGF receptor-1 or Flt-1. While it has been possible to demonstrate binding of recombinant forms of VEGF-B to isolated recombinant receptor proteins using a variety of biochemical strategies, a cell based assay, where VEGF-B binds to and dimerizes cell associated receptors to trigger activation of downstream substrates and subsequently a biological response that can be quantitated, has not been available. To address this issue, the inventors used splice-overlap-PCR techniques to generate chimeric receptors consisting of the extracellular and membrane domain of VEGFR1 fused to the cytoplasmic domain of the shared receptor component gp130. Dimerization of gp130 cytoplasmic domains leads to activation of the Jak/STAT signal transduction pathway and subsequently transcription of genes that incorporate appropriate STAT binding elements within their promoter region.

The chimeric receptor was co-transfected along with a gene encoding hygromycin resistance into 293A12 cells. 293A12 are an engineered version of standard 293T cells that have been transfected with the luciferase reporter gene under the control of a STAT responsive promoter. Stimulation of these cells with cytokines that dimerize gp130, including LIF and IL-6, leads to activation of luciferase gene transcription and subsequently quantifiable luciferase reporter activity. Following selection in hygromycin resistant clones were isolated and selected for luciferase production in response to the control protein VEGF-A. VEGF-A is a commercially available cytokine related to VEGF-B that also binds to and dimerizes the VEGFR1 receptor. Resistant clones producing luciferase in response to VEGF-A were expanded, recloned and further characterized prior to analysis of VEGF-B isoforms. Analysis of the response to VEGF-A indicated an ED₅₀ at between 10-50 ng/ml of the recombinant protein.

The cloned cell line with the highest signal to background ratio in response to VEGF-A (clone 2.19.25) was selected for analysis of refolded VEGF-B isoforms Experiments demonstrated the both naturally occurring VEGF-B isoforms as well as the artificial truncated form, were able to stimulate luciferase activity. For VEGF-B₁₈₆ and the artificial truncated form in particular the dose response was identical to that of the recombinant VEGF-A. Furthermore this activity could be blocked by incorporating soluble VEGFR1-Ig chimeric (commercially available, R&D Systems) protein into the assay. These results demonstrate that the recombinant VEGF-B proteins are correctly refolded and able to dimerize their cognate receptor in a biologically appropriate manner.

Those skilled in the art will appreciate that the invention described herein is susceptible to -variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Dissen G A, Lara H E, Fabrenbach W H, Costa M E, Ojeda S R, (1994) Endocrinology 134: 1146-1154.

Folkman J & Shing Y (1992) J. Biol. Chem. 267: 10931-10934.

Koch A E, Harlow L A, Haines G K, Amento E P, Unemoti E N, Wong W L, Pope R M,

Ferrara N, (1994) J. Immunol. 152: 4149-4156.

Senger D R, Van De Water L, Brown L F, Nagy J A, Yeo K T, Yeo T K, Berse B, Jackman R W Dvorak A M, Dvorak H F (1993) Cancer Metastasis Rev. 12: 303-324.

Sharkey A M, Chamock-Jones D S, Boocock C A, Brown K D, Smith S K, (1993) J. Reprod. Fertil. 99: 609-615.

Sunderkotter C, Steinbrink K, Goebeler M, Bhardway R, Sorg E, (1993) J. Leukocyt, Biol. 55:410-422.

Yan Z, Weich H A Bemart W, Breckwoldt M, Neulen J, (1993) J. Clin. Endocrinol. Metab. 77:1723-1725.

1. A method of purifying a peptide, polypeptide or protein from a biological sample wherein said method comprises subjecting said biological sample to affinity chromatography comprising an affinity matrix which has affinity for an N-terminal or C-terminal region of said peptide, polypeptide or protein but substantially not for the N-terminal or C-terminal region of a truncated or clipped form of said peptide, polypeptide or protein, said affinity chromatography being under chromatographic conditions sufficient to permit binding or association of full length but not truncated or non-full length peptide, polypeptide or protein, and then eluting the bound or otherwise associated peptide, polypeptide or protein from the affinity matrix and collecting same.

2. A method according to claim 1 comprising subjecting said biological sample to a first affinity chromatography comprising an affinity matrix which binds or associates said peptide, polypeptide or protein based on affinity to an N-terminal or C-terminal portion of said molecule, eluting off said bound or otherwise associated peptide, polypeptide or protein and subjecting same to a second affinity chromatography based on affinity to the other of an N-terminal or C-terminal portion of said molecule and eluting the peptide, polypeptide or protein bound or associated following said second affinity chromatography and collecting same.

3. A method according to claim 1 or 2 comprising subjecting said biological sample to an optional first affinity chromatography comprising an affinity matrix which binds or associates said peptide, polypeptide or protein based on affinity to an N-terminal or C-terminal portion of said molecule, eluting off said bound or otherwise associated peptide, polypeptide or protein and subjecting same to cation exchange chromatography and eluting the peptide, polypeptide or protein bound or associated following said cation exchange chromatography and collecting same.

4. A method according to any one of claims 1 to 3 wherein said first Unity chromatographic step is based on a polymer of basic amino acids.

5. A method according to claim 4 wherein the polymer of basic amino acids comprises polyHis or hexa-His residues.

6. A method according to claim 5 wherein the second affinity chromatographic step is based on an inherent heparin binding property of the peptide, polypeptide or protein.

7. A method according to any one of claims 1 to 6 wherein the peptide, polypeptide or protein is in recombinant form.

8. A method according to claim 7 wherein the peptide, polypeptide or protein is a VEGF-B isoform.

9. A method according to claim 8 wherein the VEGF-B isoform is VEGF-B_{167.}

10. A method according to claim 8 wherein the VEGF-B isoform is VEGF-B₁₈₆.

11. A method according to claim 8 wherein the VEGF-B isoform is VEGF-B₁₀₋₁₀₈.

12. A method according to any one of claims 8 to 11 wherein the VEGF-B isoform is tagged with hexa-His residues.

13. A method according to any one of claims 8 to 12 wherein the VEGF-B isoform is of human origin.

14. A method of a purifying full length VEGF-B isoform or a related polypeptide from a biological sample, said method comprising subjecting said biological sample to a first optional affinity chromatography comprising an affinity matrix based on affinity binding to multiple contiguous exogenous His residues in the N-terminal portion of said VEGF-B isoform, eluting said VEGF-B isoform bound or otherwise associated with said first affinity chromatography and subjecting said eluted VEGF-B isoform to a second affinity chromatography based on affinity of the C-terminal portion of said VEGF-B isoform to heparin or like molecule, and then eluting and collecting said VEGF-B isoform bound or otherwise associated by said second affinity chromatography.

15. A method of purifying a full length VEGF-B isoform or a related polypeptide from a biological sample, said method comprising subjecting said biological sample to a first optional affinity chromatography comprising an affinity matrix based on affinity binding to multiple contiguous exogenous histidine (His) residues in the N-terminal portion of said VEGF-B isoform, eluting said VEGF-B isoform bound or otherwise associated with said fist affinity chromatography and subjecting said eluted VEGF-B isoform to a cation exchange chromatography, and then eluting and collecting said VEGF-B isoform bound or otherwise associated by said cation exchange chromatography.

16. A method according to claim 14 or 15 wherein the VEGF-B isoform is VEGF-B₁₆₇.

17. A method according to claim 14 or 15 wherein the VEGF-B isoform is VEGF-B₁₈₆.

18. A method according to claim 14 or 15 wherein the VEGF-B isoform is VEGF-B₁₀₋₁₀₈.

19. A method according to any one of claims 14 to 18 wherein the VEGF-B isoform is of human origin.

20. A method according to claim 1 or 14 or 15 wherein the purified peptide, polypeptide or protein is subjected to refolding conditions in the presence of GdCl.

21. A method according to claim 1 or 14 or 15 wherein the purified peptide, polypeptide or protein is subjected to refolding conditions in the presence of arginine.

22. A method according to claim 20 or 21 wherein the peptide, polypeptide or protein is subjected to cleavage conditions after refolding but prior to purification in order to remove one or more basic amino acid residues in its N-terminal region.

23. A method according to claim 22 wherein the basic amino acid residues comprise polyHis or hexa-His.

24. A method of purifying a homomultimeric polypeptide or similar molecule from a biological sample, said method comprising subjecting said biological sample to an optional first affinity chromatography based on affinity for exogenous basic amino acids such as polyHis or hexa-His in the N-terminal portion of said polypeptide; eluting and collecting fractions containing said polypeptide, subjecting said polypeptide to a second affinity chromatography based on affinity to heparin of the C-terminal portion-of said polypeptide; eluting and collecting said polypeptide; subjecting said polypeptide to refolding conditions in the presence of GdCl or arginine and dialyzing the refolded polypeptide against acetic acid and/or other acid with similar properties; and purifying said refolded polypeptide by reversed phase chromatography.

25. A method of purifying a homomultimeric polypeptide or similar molecule from a biological sample, said method comprising subjecting said biological sample to an optional first affinity chromatography based on affinity for exogenous basic amino acids such as polyHis or hexa-His in the N-terminal portion of said polypeptide; eluting and collecting fractions containing said polypeptide, subjecting said polypeptide to cation exchange chromatography, eluting and collecting said polypeptide; subjecting said polypeptide to refolding conditions in the presence of GdCl or arginine and dialyzing the refolded polypeptide against acetic acid and/or other acid with similar properties; and purifying said refolded polypeptide by reversed phase chromatography.

26. A method according to claim 24 or 25 wherein post refolding but prior to purification, the peptide, polypeptide or protein is subjected to cleavage conditions to remove one or more exogenous basic amino acids such as polyHis or hexa-His from the N-terminal portion of said peptide, polypeptide or protein.

27. A method according to claim 24 or 25 or 26 wherein the peptide, polypeptide or protein is a VEGF-B isoform.

28. A method accord-mg to claim 27 wherein the VEGF-B isoform is VEGF-B₁₆₇.

29. A method according to claim 27 wherein the VEGF-B isoform is VEGF-B₁₈₆.

30. A method according to claim 27 wherein the VEGF-B isoform is VEGF-B₁₀₋₁₀₈.

31. A method according to any one of claims 27 to 30 wherein the VEGF-B isoform is of human origin.

32. A method for the preparation and purification of a recombinant peptide, polypeptide or protein in homomultimeric forms said method comprising culturing a microorganism or animal cell line comprising a genetic sequence encoding a monomeric form of said peptide, polypeptide or protein under conditions sufficient for expression of said genetic sequence; obtaining cell lysate, culture supernatant fluid, fermentation fluid or conditioned medium from said microorganism or animal cell line and subjecting same to a first optional affinity chromatography step based on affinity to exogenous amino acids present in the N- or C-terminal region of said peptide, polypeptide or protein, collecting fractions containing said peptide, polypeptide or protein and subjecting said fractions to a second affinity chromatography step based on affinity to an inherent property of the amino acid sequence or structure in the C-terminal portion of said polypeptide such as binding to heparin or difference in charge; said affinity chromatography being under chromatographic conditions sufficient for full length but not truncated or non-full length peptide, polypeptide or protein to be bound or otherwise associated by said affinity chromatography; eluting and collecting said full length peptide, polypeptide or protein and subjecting same to refolding conditions in the presence of GdCl or arginine and dialysing against acetic acid or other similar acid and then purifying the refolded polypeptide by reversed phase chromatography.

33. A method according to claim 32 wherein post refolding but prior to purification, the peptide, polypeptide or protein is subjected to cleavage conditions to remove one or more exogenous basic amino acids such as polyHis or hexa-His from the N-terminal portion of said peptide, polypeptide or protein.

34. A method according to claim 32 or 33 wherein the peptide, polypeptide or protein is a VEGF-B isoform.

35. A method according to claim 34 wherein the VEGF-B isoform is VEGF-B₁₆₇.

36. A method according to claim 34 wherein the VEGF-B isoform is VEGF-B₁₈₆.

37. A method according to claim 34 wherein the VEGF-B isoform is VEGF-B₁₀₋₁₀₈.

38. A method according to any one of claims 34 to 37 wherein the VEGF-B isoform is of human origin.

39. An isolated peptide, polypeptide or protein purified by the method of any one of claims 1 or 14 or 15 or 24 or 25 or 32.

40. A composition comprising a peptide, polypeptide or protein according to claim 39.

41. An isolated peptide, polypeptide or protein according to claim 39 or a composition according to claim 40 comprising a VEGF-B isoform.

42. A method according to claim 41 wherein the VEGF-B isoform is VEGF-B₁₆₇.

43. A method according to claim 41 wherein the VEGF-B isoform is VEGF-B₁₈₆.

44. A method according to claim 41 wherein the VEGF-B isoform is VEGF-B₁₀₋₁₀₈.
